(19) United States
(12) Patent Application Publication
(10) Pub. No.: US 2026/0098300 A1
(43) Pub. Date: Apr. 9, 2026

(54) CURVED METAL NANOSTRUCTURE BASED ON BIOMOLECULES AND NANOPLASMONIC BIOSENSOR USING SAME

(71) Applicant: KOREA UNIVERSITY RESEARCH AND BUSINESS FOUNDATION, Seoul (KR)

(72) Inventors: Sang Jun SIM, Seoul (KR); So Jin SONG, Seoul (KR); Jong Uk LEE, Seoul (KR)

(73) Assignee: KOREA UNIVERSITY RESEARCH AND BUSINESS FOUNDATION, Seoul (KR)

(21) Appl. No.: 19/112,694

(22) PCT Filed: Sep. 15, 2023
(86) PCT No.: PCT/KR2023/013904

§ 371 Date: Dec. 8, 2025

(30) Foreign Application Priority Data
Sep. 19, 2022 (KR) 10-2022-0118099
Jun. 29, 2023 (KR) 10-2023-0084546

(51) Int. Cl. C12Q 1/6883

(52) U.S. Cl. CPC C12Q 1/6883; C12Q 2600/112; C12Q 2600/178

(57) ABSTRACT

The present invention relates to a curved plasmonic metal nanostructure based on biomolecules and a nanoplasmonic biosensor using same.

### TECHNICAL FIELD

The present invention relates to a curved metal nanostructure based on a biomolecule and a nanoplasmonic biosensor using the same.

### BACKGROUND ART

Recent statistics estimate that approximately 50 million people worldwide suffer from dementia, and that this number is expected to double every 20 years, reaching approximately 152 million cases by 2050. In particular, Alzheimer's disease (AD) is the most common neurodegenerative disease in the elderly, characterized by progressive cognitive and functional decline, and its main symptoms are memory loss and the inability to form new memories. In particular, Alzheimer's disease is a disease from which recovery is impossible and whose progression can only be slowed down, when it has progressed beyond a certain level, and thus early diagnosis and determination of the severity of the disease are very important.

To date, Alzheimer's disease has been reported to be caused by various complex pathological and physiological conditions such as amyloid cascade, tau phosphorylation, neurotransmitters, excitotoxicity, or oxidative stress. Accordingly, it is very difficult to prevent the onset or progression of Alzheimer's disease, and thus the focus has now shifted to early diagnosis and treatment to slow its progression and ameliorate cognitive decline.

The main pathological hallmarks of Alzheimer's disease (AD) are cerebral accumulation of β-amyloid (Aβ) peptides or tau neurofibrillary tangles (NFTs), and are closely related to neurodegenerative mechanisms leading to the toxicity and destruction of neurons and synapses during pathogenesis of the disease.

Current diagnostic methods include brain imaging, cognitive function tests, and tests using cerebrospinal fluid, but these methods are insufficient for early diagnosis because they can only be applied after Alzheimer's disease symptoms have significantly developed. Additionally, the testing of these biomarker has limitations in that it requires high diagnostic costs and is not always practicable due to potential risks associated with invasive procedures.

On the other hand, blood-based diagnostics may help overcome these shortcomings because they are noninvasive, inexpensive, and enable multiple sampling even in large cohorts. Accordingly, many recent studies have investigated the correlation between blood-based Alzheimer's disease biomarkers and brain pathological changes. However, despite these efforts, there is still a lack of testing methods based on blood-based Alzheimer's disease biomarkers that can directly reflect the onset and progression of Alzheimer's disease.

Meanwhile, exosomes are small vesicles, ranging in size from 30 to 100 nm, that are released from various types of cells, including those of the central nervous system (CNS), and they have been reported to play a role in the spreading of pathogenic proteins and the aggregation of proteins such as Aβ, tau, and prions in the brain. In addition, since exosomes can pass through the blood-brain barrier (BBB) while carrying various genetic materials (DNA, miRNA, proteins, etc.) related to neural function, information about the brain status can be obtained more easily. In particular, because exosomal miRNAs (exo-miRs) participate in various fundamental processes in the central nervous system, such as neuronal differentiation, development, and functionality of mature neurons, they can provide accurate information on various characteristics of Alzheimer's disease according to disease progression. Therefore, exo-miRs are highlighted as novel diagnostic and therapeutic biomarkers for AD. However, the concentrations of blood-based biomarkers are 10 to 100 times lower than those found in cerebrospinal fluid, and blood also contains many other interfering substances. Thus, it is necessary to develop an accurate and reliable method to detect these biomarkers.

Furthermore, quantitative reverse transcription polymerase chain reaction (qRT-PCR) and microarray techniques have recently been used to detect exo-miRs. However, these techniques are mainly based on fluorescence, they have limitations in that detection reliability and sensitivity are reduced due to problems associated with fluorescent dyes (e.g., photobleaching and blinking). In addition, there are limitations in that it is not easy to obtain accurate analysis results due to the characteristics of miRNAs (e.g., short length in body fluids, high homology, low expression level, etc.).

Recently, single plasmonic nanoparticle (NP)-based sensors have attracted increasing attention as an alternative to overcome these limitations due to their ability to interact with incident light to generate localized surface plasmon resonance (LSPR). This physical phenomenon is represented by converting the changes in the refractive index (RI) around the NP into shifts in the plasmonic bands of the absorption and scattering spectra, and this allows single NP to be ultra-sensitive to small-sized molecular binding events. In addition, since the LSPR phenomenon is highly dependent on the shape, size, local RI, and array of the nanoparticles, the sensitivity of the LSPR-biosensor based on nanoparticles can be easily improved by adjusting these factors. Reflecting this trend, several recent studies have introduced LSPR-based sensors for miRNA detection. However, due to insufficient detection sensitivity and low reproducibility and selectivity of exo-miRs, their use in clinical applications is still limited, and studies are needed to overcome this limitation.

### DISCLOSURE

### Technical Problem

The present invention has been made in order to solve the above-mentioned problems, and the present invention is intended to provide a biosensor based on a curved plasmonic metal nanostructure and a method capable of accurately and simultaneously detecting exo-miRs or exosomal amyloid beta 40 and amyloid beta 42 biomarkers of Alzheimer's disease or mild cognitive impairment in isolated serum even at a very low detection limit by using the same.

### Technical Solution

One aspect of the present invention relates to a curved plasmonic metal nanostructure.

In one embodiment, the curved plasmonic metal nanostructure may comprise a first metal nanoparticle, a second metal nanoparticle, and a third metal nanoparticle, wherein the metal nanoparticles are arranged so that the angle between a first imaginary straight line connecting the midpoint of the second metal nanoparticle to the midpoint of the first metal nanoparticle and a second imaginary straight line connecting the midpoint of the second metal nanoparticle to the midpoint of the second metal nanoparticle is 125 to 135°, so that a nanocavity surrounded by the first metal nanoparticle, the second metal nanoparticle, and the third metal nanoparticle is formed, and nanogaps are formed between the first metal nanoparticle and the second metal nanoparticle and between the second metal nanoparticle and the third metal nanoparticle, respectively.

In another embodiment, the nanogaps may have a diameter of 8 to 10 nm.

In still another embodiment, the metal nanoparticles may have a diameter of 13 to 20 nm.

In yet another embodiment, the second metal nanoparticle may have single-stranded DNAs conjugated to both sides of the nanoparticle, and the first and third metal nanoparticles may have conjugated thereto a single-stranded DNA complementary to the single-stranded DNA conjugated to the second metal nanoparticle.

In still yet another embodiment, the nanostructure may be for a biosensor.

In a further embodiment, the metal may be any one selected from the group consisting of gold (Au), silver (Ag), copper (Cu), platinum (Pt), and palladium (Pd).

Another aspect of the present invention relates to a biosensor based on a curved plasmonic nanostructure.

In one embodiment, the biosensor may comprise: a substrate; a curved plasmonic nanostructure immobilized on the substrate and having a capture probe conjugated thereto that specifically binds to a target biomarker; and a measurement unit configured to measure a localized surface plasmon resonance phenomenon in the curved plasmonic nanostructure.

In another embodiment, the target biomarker may be any one or more selected from the group consisting of DNA, miRNA, a peptide, and a protein.

In still another embodiment, the miRNA may be derived from exosomes.

In yet another embodiment, the miRNA may be exo-miR125b, exo-miR135a, exo-miR15a, exo-miR20a, or a combination thereof.

In still yet another embodiment, the biosensor may detect exosome-derived miRNA and protein by measuring a Rayleigh scattering spectral change caused by specific binding of the exosome-derived miRNA and protein.

In a further embodiment, the biosensor may have no change in the Rayleigh scattering spectrum when exo-miR125b, exo-miR135a, exo-miR15a or exo-miR20a has a single point mutation.

In another further embodiment, the protein may be amyloid β (Aβ).

In still another further embodiment, the amyloid β may be any one or more selected from the group consisting of amyloid β (Aβ) 40 and amyloid beta 42.

In yet another further embodiment, the biosensor may be for diagnosing a neurodegenerative disease.

In still yet another further embodiment, the neurodegenerative disease may be Alzheimer's disease or mild cognitive impairment.

In a still further embodiment, the capture probe may comprise DNA, LNA, or an antibody.

Still another aspect of the present invention relates to a method for detecting an exosomal miRNA and protein derived from a neurodegenerative disease, comprising a step of treating the biosensor with a neurodegenerative disease-derived biomarker mixture.

In one embodiment, the biomarker mixture may be serum, blood, or plasma from which agglutinins have been removed.

In another embodiment, the neurodegenerative disease may be Alzheimer's disease or mild cognitive impairment.

In still another embodiment, the exosomal miRNA may be any one or more selected from the group consisting of exo-miR125b, exo-miR135a, exo-miR15a, and exo-miR20a.

In yet another embodiment, the protein may be amyloid β (Aβ).

In still yet another embodiment, the amyloid β may be any one or more selected from the group consisting of amyloid β (Aβ) 40 and amyloid beta 42.

Yet another aspect of the present invention relates to a method for diagnosing a neurodegenerative disease, comprising steps of: treating the biosensor with a neurodegenerative disease-derived biomarker mixture; and treating the biosensor with an exosome-derived miRNA or protein detection probe.

In one embodiment, the detection probe may comprise DNA, LNA or an antibody.

Still yet another aspect of the present invention relates to a method for distinguishing between neurodegenerative diseases.

In one embodiment, the method for distinguishing between neurodegenerative diseases may comprise steps of: treating the biosensor with a neurodegenerative disease-derived biomarker mixture; and treating the biosensor with an exosome-derived miRNA or protein detection probe.

In another embodiment, the method may be a method for distinguishing between Alzheimer's disease and mild cognitive impairment.

A further aspect of the present invention relates to a method for fabricating a curved plasmonic nanostructure.

In one embodiment, the method for fabricating a curved plasmonic nanostructure may comprise steps of: (a) incubating metal nanoparticles with one single-stranded DNA; (b) the isolating metal nanoparticles conjugated with the one single-stranded DNA; (c) hybridizing the metal nanoparticles conjugated with the one single-stranded DNA to metal nanoparticles conjugated with two single-stranded DNAs, which have a DNA sequence complementary to that of the metal nanoparticles conjugated with the one single-stranded DNA, at a ratio of 2:1, and then isolating the hybridized metal seeds; (d) coating the metal seeds; and (e) crystallizing the coated metal seeds by treatment with a metal precursor and a reducing agent in the presence of sodium chloride.

In another embodiment, the sodium chloride may be used at a concentration of 50 to 100 mM.

In still another embodiment, the fabricated nanostructure may comprise three metal nanoparticles in which two single-stranded DNAs are bound with one single-stranded DNA having a sequence complementary thereto by treatment with sodium chloride, and the three metal nanoparticles are arranged so that the angle between imaginary straight lines connecting the midpoints of the three metal nanoparticles is 125 to 135°, so that the nanostructure includes a nanocavity surrounded by the three metal nanoparticles.

Another further aspect of the present invention relates to a method for fabricating a curved plasmonic nanostructure-based biosensor.

In one embodiment, the method may comprise steps of: (a) incubating metal nanoparticles with one single-stranded DNA; (b) the isolating metal nanoparticles conjugated with the one single-stranded DNA; (c) hybridizing the metal nanoparticles conjugated with the one single-stranded DNA to metal nanoparticles conjugated with two single-stranded DNAs, which have a DNA sequence complementary to that of the metal nanoparticles conjugated with the one single-stranded DNA, at a ratio of 2:1, and then isolating the hybridized metal seeds; (d) coating the metal seeds; (d) coating the metal seeds; (e) crystallizing the coated metal seeds by treatment with a metal precursor and a reducing agent in the presence of sodium chloride to obtain a curved plasmonic nanostructure; (f) immobilizing the curved plasmonic nanostructure onto a substrate; and (g) conjugating a capture probe, which specifically binds to an isolated target biomarker, to the curved plasmonic nanostructure.

In another embodiment, the coating may be made with polyethylene glycol (PEG).

In still another embodiment, the method may further comprise, before step (d), a step of coating the substrate with (3-mercaptopropyl) trimethoxysilane (MP TES) 3- or aminopropyltriethoxysilane (APTES).

In yet another embodiment, the crystallization may be performed at pH 5.

In still yet another embodiment, the biosensor may be for diagnosing a neurodegenerative disease.

### Advantageous Effects

According to one embodiment of the present invention, neurodegenerative disease markers present in blood, for example, exosome-derived miRNAs and/or proteins, may be detected with high selectivity and sensitivity, and thus may be effectively used for the diagnosis of miRNA or protein-related diseases and in clinical applications.

### BRIEF DESCRIPTION OF DRAWINGS

FIG. 1 shows the design of programmable curved plasmonic nanostructures with enhanced optical properties according to one embodiment of the present invention. Specifically, a) shows Au nanotrimer gaps (top panel left; 180°, top panel middle; 130°, and top panel right; 70°) and Au nanoarchitectures (bottom panel left; 180°, bottom panel middle; 130°, and bottom panel right, 70°), and b) shows the results of measuring refractive index sensitivity for Au nanotrimer gaps (top panel left; 180°, top panel middle; 130°, and top panel right; 70°) and Au nanoarchitectures (bottom panel left; 180°, bottom panel middle; 130°, and bottom panel right, 70°).

FIG. 2 shows programmable curved plasmonic architecture seeds prepared according to one embodiment of the present invention. a) shows single-stranded DNA-conjugated gold nanoparticles, and b) shows architecture seeds separated by gel electrophoresis after hybridization.

FIG. 3 shows the synthesis and characterization of programmable curved plasmonic nanostructures according to one embodiment of the present invention. Specifically, a) is a schematic diagram showing a crystallization mechanism using a direction-specific crystallization method. b) shows EF-TEM images of novel synthesized nanostructures depending on the NaCl concentration, c) is an EF-TEM image of a programmable curved plasmonic nanostructure synthesized at pH 5 and an NaCl concentration of 75 mM, and d) is an EF-TEM image and FFT pattern of a programmable curved plasmonic nanostructure synthesized by the DNA-directed crystallization technique, where the spacing of crystal lattice planes is 0.2045±0.0005 nm, as indicated by arrows. e) shows the size distributions of the diameter (left), nanogap length (middle), and nanocavity angle (right) of programmable curved plasmonic nanoarchitectures analyzed by Image J, and shows the statistical results of 100 nanoarchitectures. f) shows the resonant Rayleigh scattering spectra of the programmable curved plasmonic nanostructure, experimentally observed (left, n=100) and theoretically calculated (right), and the inset is a dark-field image of a single nanoarchitecture. g) shows the charge density distribution of the programmable curved plasmonic nanostructure upon excitation at 529 nm.

FIG. 4 shows the results of measuring zeta potential (a) and dynamic light scattering (DLS) (b) depending on the concentration of sodium chloride (NaCl) to examine the growth pattern of curved particles having a gap space according to one embodiment of the present invention.

FIG. 5 is a schematic diagram showing the configuration of an LSPR system according to one embodiment of the present invention.

FIG. 6 shows a fabricated programmable curved plasmonic nanoarchitecture-based plasmonic biosensor for diagnosing Alzheimer's disease according to one embodiment of the present invention. a) is a schematic diagram showing the detection of target exomiRs (exomiR-125b, exomiR-135a, exomiR-15a, and exomiR-20a) using the programmable curved biosensor, and b) shows nanostructure-based optical representative Rayleigh light scattering spectra of individual programmable curved nanoarchitectures depending on target exomiR.

FIG. 7 shows the results of verifying the sensitivity and selectivity of a sensor according to one embodiment of the present invention. It shows that the LSPR peak shifts in response to detection of various concentrations of exomiR-125b (a), exomiR-135a (b), exomiR-15a (c), and exomiR-20a (d) in the concentration range of 10^{-0.5} to 10^{9.5} aM. It shows strong linearity between the LSPR peak shifts of exomiR-125b (a), exomiR-135a (b), exomiR-15a (c), and exomiR-20a (d) in the concentration range of and 10^{0.5} to 10^{8.5} aM. For exomiR-125b (e), exomiR-135a (f), exomiR-15a (g) and exomiR-20a (h), three types of miRNA sequences containing type A (3′ end mutation), type B (middle site mutation), and type C (5′ end mutation) single point mutations were used to test the sensor specificity. The probe and miRNA sequences are detailed in Table 2 below.

FIG. 8 shows an HR-TEM image of serum-derived exosomes according to one embodiment of the present invention.

FIG. 9 is box plots of AD biomarkers detected in clinical samples according to one embodiment of the present invention, and shows the estimated LSPR signals of exomiR-125b (a), exomiR-135a (b), exomiR-15a (c), and exomiR-20a (d) from sera of HCs (n=5), AD patients (n=24), and MCI patients (n=14).

FIG. 10 is box plots of AD biomarker detection in clinical serum samples according to one embodiment of the present invention, and shows the expression levels of exomiR-125b (a), exomiR-135a (b), exomiR-15a (c), and exomiR-20a (d) in serum samples from HCs (n=5), AD patients (n=24), and MCI patients (n=14). The levels of AD biomarkers were analyzed by qRT-PCR.

FIG. 11 shows receiver operating characteristic curves of single AD biomarkers, exomiR-125b (a), exomiR-135a (b), exomiR-15a (c), and exomiR-20a (d), for distinguishing between AD patients (n=24) and HCs (n=5) according to one embodiment of the present invention.

FIG. 12 shows receiver operating characteristic curves of single AD biomarkers, exomiR-125b (a), exomiR-135a (b), exomiR-15a (c), and exomiR-20a (d), for distinguishing between MCI patients (n=14) and HC (n=5) according to one embodiment of the present invention.

FIG. 13 shows AD biomarkers detected in clinical samples according to one embodiment of the present invention. It shows ROC curves of AD biomarker ratios between Aβ42 and exomiR-125b (a), exomiR-135a (c), exomiR-15a (e), and exomiR-20a (g) for distinguishing between AD patients and HCs. The box plots show the estimated ratios of exomiR-125b/Aβ42 (b), exomiR-135a/Aβ42 (d), exomiR-15a/Aβ42 (f), and exomiR-20a/Aβ42 (h) extracted from the sera of AD patients (n=24) and HCs (n=5). Also, it shows ROC curves of AD biomarker ratios between Aβ42 and exomiR-125b (i), exomiR-135a (k), exomiR-15a (n), and exomiR-20a (o) for differentiating between MCI patients and HCs. The box plots show the estimated ratios of exomiR-125b/Aβ40 (j), exomiR-135a/Aβ42 (1), exomiR-15a/Aβ42 (m), and exomiR-20a/Aβ42 (p) extracted from sera of MCI patients (n=14) and HCs (n=5).

FIG. 14 shows receiver operating characteristic curves for the ratios between Aβ1-40 and exomiR-125b (a), exomiR-135a (c), exomiR-15a (e), and exomiR-20a (g) for distinguishing between AD patients (n=24) and HCs (n=5) according to one embodiment of the present invention. The box plots show the estimated ratios of exomiR-125b/Aβ40 (b), exomiR-135a/Aβ40 (d), exomiR-15a/Aβ40 (f), and exomiR-20a/Aβ40 (h) in serum samples from AD patients (n=24) and HCs (n=5).

FIG. 15 shows receiver operating characteristic curves for the ratios between Aβ1-40 and exomiR-125b (a), exomiR-135a (c), exomiR-15a (e) and exomiR-20a (g) for distinguishing between MCI patients (n=14) and HCs (n=5) according to one embodiment of the present invention. The box plots show the estimated ratios of exomiR-125b/Aβ40 (b), exomiR-135a/Aβ40 (d), exomiR-15a/Aβ40 (f) and exomiR-20a/Aβ40 (h) in serum samples from MCI patients (n=14).

FIG. 16 shows receiver operating characteristic curves for the ratios between Aβ1-40 and exomiR-125b (a), exomiR-135a (c), exomiR-15a (e), and exomiR-20a (g) for distinguishing between MCI patients (n=14) and AD patients (n=24) according to one embodiment of the present invention. The box plots show the estimated ratios of exomiR-125b/Aβ40 (b), exomiR-135a/Aβ40 (d), exomiR-15a/Aβ40 (f), and exomiR-20a/Aβ40 (h) in serum samples from MCI patients (n=14) and AD patients (n=24).

FIG. 17 shows receiver operating characteristic curves for the ratios between Aβ1-42 and exomiR-125b (a), exomiR-135a (c), exomiR-15a (e), and exomiR-20a (g) for distinguishing between MCI patients (n=14) and AD patients (n=24) according to one embodiment of the present invention. The box plots show the estimated ratios of exomiR-125b/Aβ42 (b), exomiR-135a/Aβ42 (d), exomiR-15a/Aβ42 (f), and exomiR-20a/Aβ42 (h) in serum samples from MCI patients (n=14) and AD patients (n=24).

The present invention will be described in detail below. Meanwhile, each description and embodiment disclosed in the present invention may also be applied to each other description and embodiment. That is, all combinations of various elements disclosed in the present invention fall within the scope of the present invention. In addition, the scope of the present invention may not be considered to be limited by the specific description described below.

Furthermore, those skilled in the art will recognize, or be able to ascertain, using no more than routine experimentation, numerous equivalents to the specific embodiments described herein. Such equivalents are also intended to be encompassed by the present invention.

The present invention has been made in order to solve the problem that conventional Western blotting or qRT-PCR has limitations in detecting target biomarkers related to Alzheimer's disease.

Therefore, in one aspect, the present invention provides a curved plasmonic metal nanostructure comprising a first metal nanoparticle, a second metal nanoparticle, and a third metal nanoparticle, wherein the metal nanoparticles are arranged so that the angle between a first imaginary straight line connecting the midpoint of the second metal nanoparticle to the midpoint of the first metal nanoparticle and a second imaginary straight line connecting the midpoint of the second metal nanoparticle to the midpoint of the second metal nanoparticle is 125 to 135°, so that a nanocavity surrounded by the first metal nanoparticle, the second metal nanoparticle, and the third metal nanoparticle is formed, and nanogaps are formed between the first metal nanoparticle and the second metal nanoparticle and between the second metal nanoparticle and the third metal nanoparticle, respectively.

In another aspect, the present invention provides a nanoplasmonic biosensor based on the curved plasmonic metal nanostructure.

Specifically, the biosensor may comprise: a substrate; a curved plasmonic nanostructure immobilized on the substrate and having a capture probe conjugated thereto that specifically binds to a target biomarker; and a measurement unit configured to measures a localized surface plasmon resonance phenomenon in the curved plasmonic nanostructure.

Hereinafter, the present invention will be described in more detail.

In the present invention, the term “nanoplasmonic biosensor” refers to a biosensor that can measure plasmons, in which the plasmon refers to an electron or a quantum of valence oscillation, i.e. plasma oscillation. That is, the nanoplasmonic biosensor refers to a biosensor comprising a measurement unit capable of measuring plasmons, which are pseudo-particles in which free electrons in metals oscillate collectively.

The biosensor may be based on the curved plasmonic nanostructure.

In the present invention, the term “curved plasmonic metal nanostructure” means a plasmonic metal nanostructure in which three advanced plasmonic metal nanoparticles conjugated with DNA are combined to form a trimeric structure, and the trimeric structure has a shape curved at a specific angle.

Specifically, the curved plasmonic metal nanostructure may comprise a first metal nanoparticle, a second metal nanoparticle, and a third metal nanoparticle, wherein the metal nanoparticles are arranged so that the angle between a first imaginary straight line connecting the midpoint of the second metal nanoparticle to the midpoint of the first metal nanoparticle and a second imaginary straight line connecting the midpoint of the second metal nanoparticle to the midpoint of the second metal nanoparticle is 125 to 135°, so that a nanocavity surrounded by the first metal nanoparticle, the second metal nanoparticle, and the third metal nanoparticle is formed, and nanogaps are formed between the first metal nanoparticle and the second metal nanoparticle and between the second metal nanoparticle and the third metal nanoparticle, respectively.

The term “nanogap” means a nanometer-sized gap, and specifically means a gap formed between two metal nanoparticles when they are connected in a bridge form, and may be used interchangeably with the term “nanocrevice”. The nanogap is different from an intergap formed when metal nanoparticles are parallel and partially overlap each other.

The nanogaps may have a diameter of 6 to 12 nm, specifically 8 to 10 nm, without being limited thereto.

The curved plasmonic metal nanostructure of the present invention may include two nanogaps between the first metal nanoparticle and the second metal nanoparticle and between the second metal nanoparticle and the third metal nanoparticle, respectively, because the three metal nanoparticles have a shape curved at a specific angle.

The term “nanocavity” refers to a nanospace created when the three metal nanoparticles are arranged at a specific angle.

Specifically, the nanocavity may be formed to be surrounded by the first metal nanoparticle, the second metal nanoparticle, and the third metal nanoparticle, because the nanoparticles are arranged so that the angle between a first imaginary straight line connecting the midpoint of the second metal nanoparticle located at the center to the midpoint of the first metal nanoparticle and a second imaginary straight line connecting the midpoint of the second metal nanoparticle to the midpoint of the second metal nanoparticle is 125 to 135°.

The term “metal nanoparticle” means a nanometer-sized particle made of a metal, and the diameter of the metal nanoparticle may be 13 to 20 nm, specifically 14 to 16 nm. The term “metal nanoparticle” may be used interchangeably with the term “nanosphere”.

The metal may be any one selected from the group consisting of gold (Au), silver (Ag), copper (Cu), platinum (Pt), and palladium (Pd), without being limited thereto.

The second metal nanoparticle may have single-stranded DNAs conjugated to both sides of the nanoparticle, and the first and third metal nanoparticles may have conjugated thereto a single-stranded DNA complementary to the single-stranded DNA conjugated to the second metal nanoparticle.

In addition, the curved plasmonic metal nanostructure may comprise one single-stranded DNA and two single-stranded DNAs having a sequence complementary thereto at a ratio of 2:1.

The one single-stranded DNA refers to DNA consisting of one strand, and “the nanoparticle having one single-stranded DNA conjugated thereto” refers to a nanoparticle having one single-stranded DNA conjugated to one side of the nanoparticle.

The two single-stranded DNAs include two DNAs, each consisting of one strand, and refer to two single-stranded DNAs conjugated to both sides of the nanoparticle, specifically, at an angle of 125 to 135°. In this case, the one single-stranded DNA and the two single-stranded DNAs may have complementary sequences so as to be able to hybridize with each other.

The nanoparticles having one single-stranded DNA conjugated thereto and the nanoparticles having the two single-stranded DNAs conjugated thereto may be present at a ratio of 2:1. In this case, one single-stranded DNA and one strand of two single-stranded DNAs may complementarily bind to each other, and another single-stranded DNA and the other strand of the two single-stranded DNAs may complementarily bind to each other.

As a result, three nanoparticles are hybridized through complementary binding between the DNAs, and the three nanoparticles (nanospheres) form a bridged structure, but may have a shape curved at a specific angle of 125 to 135° rather than a parallel structure with an angle of 180°.

The DNAs may be used without limitation as long as they may complementarily bind to each other, and the length thereof may be, for example, 75 bp to 150 bp, for another example, 85 bp to 120 bp, and for another example, 100 bp, without being limited thereto.

The curved plasmonic metal nanostructure comprises three nanoparticles, each having a single-stranded DNA sequence, wherein nanoparticles each having one single-stranded DNA are hybridized to both sides of the second nanoparticle having two single-stranded DNAs by complementary binding, and then the nanoparticles are crystallized at pH 5 in a DNA-directed manner, and in particular, a structure having a specific angle of 125 to 135° C. is formed by treatment with sodium chloride.

The curved plasmonic metal nanostructure may be conjugated with a capture probe that specifically binds to a target biomarker.

In the present invention, the term “target biomarker” means a biomarker to be detected or diagnosed in an isolated form obtained from a living body. For example, the target biomarker may be any one or more selected from the group consisting of DNA, miRNA, a peptide, and a protein, and as another example, may be miRNA, a protein, or a combination thereof, without being limited thereto.

miRNA is a microRNA, a short (20-24 nt) noncoding RNA that affects both the stability and translation of mRNA and is involved in the post-transcriptional regulation of gene expression in multicellular organisms. For example, the miRNA may be derived from exosomes, and as another example, may be exosome-derived miRNA obtained from individuals with Alzheimer's disease or mild cognitive impairment.

The exosomes are a type of extracellular vesicles (EVs) that are produced within cells and released to the outside, and are vesicles with a size of 50 to 150 nm that are secreted through information exchange between cells in eukaryotes.

The exosome-derived miRNA may be exo-miR125b, exo-miR135a, exo-miR15a, exo-miR20a, or a combination thereof, without being limited thereto.

The protein may be amyloid β (Aβ).

The amyloid β is a 36-43 aa peptide, the main component of amyloid plaques found in the brains of Alzheimer's disease (AD) patients. Since amyloid plaques are closely related to Alzheimer's disease, they are classified as Alzheimer's disease biomarkers. The amyloid β may include amyloid β1 to 42, and as a specific example, may be amyloid beta 40, amyloid beta 42, or a combination thereof.

In another embodiment, the target biomarker may include a combination of exo-miR and amyloid beta.

In another embodiment, the target biomarker may be exo-miR125b, exo-miR135a, exo-miR15a, exo-miR20a, amyloid beta 40, amyloid beta 42, or a combination thereof.

The biosensor may detect exosome-derived miRNA and protein by measuring a Rayleigh scattering spectral change caused by specific binding of the exosome-derived miRNA and protein.

In particular, the biosensor may have no change in the Rayleigh scattering spectrum when exo-miR125b, exo-miR135a, exo-miR15a or exo-miR20a has a single point mutation.

The biosensor may be for diagnosing a neurodegenerative disease.

The term “neurodegenerative disease” includes, in a broad sense, all pathological conditions in which nerve cells degenerate, but generally does not include those that can be caused by distinct nervous cells, such system as cerebrovascular damage, trauma, and metabolic disorders. Specifically, the above neurodegenerative disease may be Alzheimer's disease or mild cognitive impairment, without being limited thereto.

The term “Alzheimer's disease (AD)” refers to a disease reported to be caused by various complex pathological and physiological conditions, including amyloid cascade, tau phosphorylation, neurotransmitters, excitotoxicity, or oxidative stress. The main physiological hallmarks of Alzheimer's disease (AD) are cerebral accumulation of β-amyloid (Aβ) peptides or tau neurofibrillary tangles (NFTs), and are closely related to neurodegenerative mechanisms leading to the toxicity and destruction of neurons and synapses during pathogenesis of the disease.

The term “mild cognitive impairment (MCI)” refers to a state in which cognitive function is lower than that of the same age, but independence in activities of daily living is preserved. Mild cognitive impairment is not yet dementia. This state must not meet the diagnostic criteria for delirium or other: psychiatric disorders. In other words, mild cognitive impairment is a high-risk state for dementia. In the case of normal elderly people, only 1 to 2% progress to dementia each year, but in the case of people with mild cognitive impairment, about 10 to 15% progress to dementia each year. In addition, this state is known to be clinically important in that it is the stage where dementia can be detected at the earliest stage and where the treatment effect can be maximized.

The term “capture probe” may be used interchangeably with the term “binding probe”, etc. The capture probe may specifically bind to a target or target biomarker to be detected.

The capture probe may comprise DNA, LNA or an antibody.

The term “LNA” was introduced to further improve the sensitivity and selectivity of the sensor, and may include a moiety of a chimeric ribose ring fixed between 2′-oxygen and 4′-carbon via an O-methylene bridge. This LNA structure is capable of improving the hybridization specificity, duplex stability, and binding affinity of the biosensor.

In one specific example of the present invention, it was confirmed that a target exo-miRNA having a complementary sequence that is completely identical to a probe comprising two LNAs bound to the curved plasmonic nanostructure-based biosensor of the present invention, and thus the sandwich structure was stably maintained, thereby stably providing a detection signal.

The biosensor may detect exosome-derived miRNA and protein by measuring a Rayleigh scattering spectral change caused by specific binding of the exosome-derived miRNA.

In addition, the biosensor may detect exosome-derived miRNA in a very low concentration range of attomolar.

According to one embodiment of the present invention, the biosensor may detect exosome-derived miRNA even at a low detection limit, without being limited thereto.

The term “substrate” refers to a plate on which the metal nanoparticle platform may be immobilized so that observation can be performed under a microscope or the like. Specifically, the substrate may be a glass slide, without being limited thereto.

In one specific example of the present invention, it was confirmed that a curved plasmonic metal nanostructure-based biosensor fabricated by immobilizing a fabricated curved plasmonic metal nanostructure on a substrate and conjugating the curved plasmonic metal nanostructure with a capture probe comprising DNA or LNA specific to an isolated target biomarker, for example, an exosome-derived miRNA or protein, was able to effectively detect even a trace amount (attomolar) through the detection probe when the target biomarker exosome-derived miRNA or protein, particularly, exo-miR125b, exo-miR135a, exo-miR15a, exo-miR20a or a combination thereof, or the protein amyloid beta 40 and/or 42, was injected thereinto.

In another aspect, the present invention provides a method for detecting an exosome-derived miRNA and protein, comprising a step of treating the curved plasmonic metal nanostructure-based biosensor with a neurodegenerative disease-derived biomarker mixture.

Here, the terms “curved plasmonic metal nanostructure-based biosensor”, “exosome”, “miRNA”, “neurodegenerative disease” and “protein” are as described above.

In the present invention, the term “biomarker mixture” means a mixture of biomarkers to be detected in the present invention. As an example, the biomarker mixture may be plasma, blood, or plasma from which agglutinins have been removed, and as another example, the biomarker mixture may contain exosome-derived miRNA or protein, or a combination thereof. As another example, the biomarker mixture may contain exosome-derived miRNA or protein or, a combination thereof isolated from an individual with Alzheimer's disease or mild cognitive impairment, and as another example, the biomarker mixture may contain exo-miR125b, exo-miR135a, exo-miR15a exo-miR20a, or a combination thereof, or a protein such as amyloid beta 40 and/or 42, but any mixture containing the miRNA or protein to be measured in the present invention may be included without limitation.

In still another aspect, the present invention provides a method for diagnosing a neurodegenerative disease, comprising steps of: treating the curved plasmonic metal nanostructure-based biosensor with a neurodegenerative disease-derived biomarker mixture; and treating the biosensor with an exosome-derived miRNA or protein detection probe.

Here, the terms “curved plasmonic metal nanostructure-based biosensor”, “exosome”, “miRNA”, “neurodegenerative disease”, “biomarker mixture” and “protein” are as described above.

In the present invention, the term “detection probe” refers to a probe capable of detecting a target miRNA or protein complementarily bound to the capture probe conjugated to the curved plasmonic metal nanostructure included in the biosensor. For example, the detection probe may be a probe capable of detecting a target miRNA or protein by complementary binding to the target miRNA or protein, and as another example, may comprise DNA or LNA, without being limited thereto.

In yet another aspect, the present invention provides a method for distinguishing between neurodegenerative diseases, comprising steps of: treating the curved plasmonic metal nanostructure-based biosensor with a neurodegenerative disease-derived biomarker mixture; and treating the biosensor with an exosome-derived miRNA or protein detection probe.

Here, the terms “curved plasmonic metal nanostructure-based biosensor”, “exosome”, “miRNA”, “neurodegenerative disease”, “biomarker mixture”, “detection probe” and “protein” are as described above.

In still yet another aspect, the present invention provides a method for fabricating a curved plasmonic nanostructure, comprising steps of: (a) incubating metal nanoparticles with one single-stranded DNA; (b) isolating the metal nanoparticles conjugated with the one single-stranded DNA; (c) hybridizing the metal nanoparticles conjugated with the one single-stranded DNA to metal nanoparticles conjugated with two single-stranded DNA-conjugated, which have a DNA sequence complementary to that of the metal nanoparticles conjugated with the one single-stranded DNA, at a ratio of 2:1, and then isolating the hybridized metal seeds; (d) coating the metal seeds; and (e) crystallizing the coated metal seeds by treatment with a metal precursor and a reducing agent in the presence of sodium chloride.

Here, the terms “curved plasmonic metal nanostructure”, “metal nanoparticles”, “metal nanoparticles conjugated with one single-stranded DNA”, “hybridization” and “metal nanoparticles conjugated with two single-stranded DNAs having a sequences complementary thereto” are as described above.

The term “coating” means covering the outer surface of a material with a specific material to form a thin layer. For example, the coating may be made with polyethylene glycol (PEG), without being limited thereto.

The term “crystallization” means crystallizing the metal seeds by AuCl⁴⁻ reducing to Au atoms during nanostructure synthesis with the metal precursor and the reducing agent. Crystallization of the metal may occur along the dsDNA strands rather than on the surface of the metal NPs.

In addition, the crystallization may be performed in an appropriate pH range so that a more curved nanostructure may be easily formed. For example, the crystallization may be performed at pH 5.

The sodium chloride may be used at a concentration of 50 to 100 mM, specifically, 70 to 80 mM. By treatment with sodium chloride in a specific concentration range, it is possible to fabricate a curved metal nanostructure comprising three metal nanoparticles in which two single-stranded DNAs are each bound to one single-stranded DNA having a sequence complementary thereto, wherein nanogaps are included between the metal nanoparticles, nanocavity is included between the three metal nanoparticles, and the nanocavity angle is 125 to 135°.

In a further aspect, the present invention provides a method for fabricating a curved plasmonic nanostructure-based biosensor, comprising steps of: (a) incubating metal nanoparticles with one single-stranded DNA; (b) isolating the metal nanoparticles conjugated with the one single-stranded DNA; (c) hybridizing the metal nanoparticles conjugated with the one single-stranded DNA to metal nanoparticles conjugated with two single-stranded DNA-conjugated, which have a DNA sequence complementary to that of the metal nanoparticles conjugated with the one single-stranded DNA, at a ratio of 2:1, and then isolating the hybridized metal seeds; (d) coating the metal seeds; (e) crystallizing the coated metal seed by treatment with a metal precursor and a reducing agent in the presence of sodium chloride to obtain a curved plasmonic nanostructure; (f) immobilizing the curved plasmonic nanostructure onto a substrate; and (g) conjugating a capture probe, which specifically binds to an isolated target biomarker, to the curved plasmonic nanostructure.

Here, the terms “curved plasmonic metal nanostructure”, “metal nanoparticles”, “metal nanoparticles conjugated with one single-stranded DNA”, “hybridization”, “metal nanoparticles conjugate with two single-stranded DNAs having a sequence complementary thereto”, “capture probe”, “coating”, “crystallization” and “substrate” are as described above.

The method may further comprise, before step (d), a step of coating the substrate with (3-mercaptopropyl) trimethoxysilane (MPTES) or 3-aminopropyltriethoxysilane (APTES).

### MODE FOR INVENTION

Hereinafter, the present invention will be described in more detail by way of examples. These examples are only for the purpose of illustrating the present invention, and it will be obvious to those skilled in the art that the scope of the present invention is not to be construed as being limited by these examples. Therefore, the substantial scope of the present invention will be defined by the appended claims and equivalents thereto.

### Experimental Example 1: Materials

Anhydrous ethyl alcohol (CH₃CH₂OH, ≥99.5%), 1,4-dithiothreitol (DTT), ethyl acetate (anhydrous, 99.8%), bis(p-sulfonatophenyl)phenylphosphine dihydrate dipotassium salt (BSPP), ethanolamine, gold (III) chloride trihydrate (299.0%), hydrochloric acid (HCl, 37 wt % in water), 3-mercaptopropyl) trimethoxysilane (MPTES), sodium dodecyl sulfate (SDS), and sodium chloride (NaCl) were purchased from Sigma Aldrich Korea, Republic of Korea). Also, methoxypolyethylene glycol-thiol (mPEG-SH) was purchased from Futurechem (Korea).

Gold nanoseed (AuNS; 15 nm) solution was purchased from British BioCell International (UK). Saline sodium citrate (SSC) buffer, 5×TBE buffer, and diethyl pyrocarbonate (DEPC)-treated water were purchased from Biosesang (Korea). Ambion™ diethylpyrocarbonate (DEPC) treated water, total exosome RNA isolation kit, TaqMan™ microRNA assay kit (has-miR-125b, has-miR-135a, has-miR-15a, has-miR-20a, and U6 snRNA), TaqMan™ Universal Master Mix II, and TaqMan™ microRNA reverse transcription kit were purchased from Thermo Fisher Scientific (USA). Coverslip slides (22×40×0.1 mm) were purchased from Deckglaser (Germany). DNA LoBind tubes and protein LoBind tubes were purchased from Eppendorf (Germany). Capture/detection DNA/LNA probes, target miRNAs, and single-point mutation miRNAs were synthesized by Macrogen (Korea).

### Experimental Example 2: Extraction of Serum-Derived Exosomal miRNAs (exomiRs)

After loading 1 mL of serum sample onto a qEV size exclusion chromatography column (Izon Science Ltd., New Zealand), exosomes were eluted with PBS buffer according to the manufacturer's protocol. The isolated serum-derived exosomes were concentrated by centrifugation at 4,000×g and 14,000×g in Amicon Ultra 15 mL and 0.5 mL centrifugal filters (Merck Millipore, USA), respectively, for 20 minutes. Thereafter, exosome-derived miRNAs were isolated using the total exosome RNA and protein isolation Kit (Invitrogen, USA) according to the manufacturer's protocol. Next, the concentration and purity of the isolated exomiRs were measured using a NanoDrop spectrophotometer (NANODROP2000, ThermoFisher Scientific, USA).

### Experimental Example 3: Quantitative Reverse Transcription Polymerase Chain Reaction (gRT-PCR) Analysis

The contents of serum-derived exosomal miRNAs from 6 healthy controls (HC) and 10 AD patients were estimated by qRT-PCR. Specifically, total RNA (2 ng/μL) was reverse transcribed into cDNA using the TaqMan miRNA reverse transcription kit (ThermoFisher Scientific, USA) at 16° C. for 30 min, 42° C. for 30 min, 85° C. for 5 min, and 4° C. for 5 min. Next, real-time qPCR was performed using a TaqMan miRNA assay kit (ThermoFisher Scientific, USA) and an Applied Biosystems StepOnePlus real-time PCR system (Applied Biosystems) for 10 min at 95° C., followed by 42 cycles, each consisting of 95° C. for 15 sec and 60° C. for 1 min. After generating the cycle threshold (Ct) values using the SDS program (v2.0.1; Applied Biosystems), the relative expression levels of the target exosomal miRNAs were normalized to the internal control (U6) and then calculated.

### Experimental Example 4: Analysis of Relative Expression Levels of Serum-Derived exomiRs by qRT-PCR

ExomiR was reverse transcribed into cDNA using 2 ng/μL of total RNA and the TaqMan miRNA reverse transcription kit (ThermoFisher Scientific, USA). The reaction was performed at 16° C. for 30 minutes, 42° C. for 30 minutes, 85° C. for 5 minutes, and 4° C. for 5 minutes. In this case, the synthesized CDNA can be stored at −20° C. for 2 months. Thereafter, qRT-PCR was performed under the conditions of 95° C. for 10 minutes, followed by 45 cycles, each consisting of 95° C. for 15 seconds and 60° C. for 1 minute. At this time, the TaqMan miRNA assay kit (ThermoFisher Scientific, USA) and the Applied Biosystems StepOnePlus real-time PCR system (Applied Biosystems) were used. The cycle threshold (Ct) value of each exomiR was calculated using the SDS program provided by ThermoFisher Scientific (v2.0.1; Applied Biosystems). Thereafter, the relative expression level of each target exomiR was normalized to U6 as an internal control and then checked.

### Experimental Example 5: Calculation of Limit of Detection (LOD)

The linear regression equations for the four AD markers are as follows:

$$exomiR - 125 b : y = 2.2 \; 92 log ( x ) + 2.205 ( {R}^{2} = 0.9 \; 94 ) ;$$

$$exomiR - 135 a : y = 2.3 \; 16 log ( x ) + 2.169 ( {R}^{2} = 0.9 \; 98 ) ;$$

$$exomiR - 15 a : y = 2.3 \; 18 log ( x ) + 1.847 ( {R}^{2} = 0.9 \; 98 ) ;$$

$$exomiR - 20 a : y = 2.3 \; 63 log ( x ) + 1.848 ( {R}^{2} = 0.997 ) .$$

In addition, the limit of detection (LOD) of the sensor was calculated using the following equation:

$$LOD = 3.3 \times δ / S$$

wherein δ is the standard deviation (SD) of the blank, S is the slope of the calibration curve. The LODs of the sensors were calculated to be 3.49 aM, 3.37 aM, 4.03 aM, and 4.01 aM for exomiR-125b, exomiR-135a, exomiR-15a, and exomiR-20a, respectively.

### Experimental Example 6: Statistical Analysis

OriginPro 8 software (OriginLab) was used to calculate the Pearson's correlation between AD-related clinical parameters (age and MMSE) and miRNA expression levels. The statistical significance of the correlation was evaluated using a standard two-tailed Student's t-test. Here, p<0.05 was considered statistically significant.

### Example 1: Pre-Design of Programmable Curved Plasmonic Nanoarchitecture Through Finite-Difference Time-Domain (FDTD) Simulation

A novel nanostructure of the present invention, which has enhanced optical properties, was pre-designed through FDTD simulation (Lumerical Inc., Vancouver, Canada).

Specifically, the novel nanostructure was designed by connecting three gold nanospheres (15.8 nm diameter) using a gold nanogap (nanocrevice, 9.5 nm diameter) with bending angles of 130° and 70°. In addition, the electric field (EF) distribution of each nanostructure was compared for two types of nanosphere trimer gap (1.0-nm gap) with bending angles of 130° and 180°, a linear-shaped respectively, and nanostructure. At this time, the wavelength range of light was 400 to 1,000 nm, and the mesh size was 0.5 nm. The scattering cross-section was calculated by changing the refractive index (RI) range of 1.33 to 1.45 covering the material range.

### Example 2: Synthesis of Pre-Designed Programmable Curved Plasmonic Nanoarchitecture

The programmable curved plasmonic nanoarchitecture pre-designed in Example 1 was synthesized as follows.

Specifically, AuNPs (diameter: 15.0 nm) were incubated with thiol-modified single-stranded DNA (ssDNA; 100 bp) overnight at 25° C. The ssDNA-conjugated gold nanoparticles were separated by gel electrophoresis at 75 V for 50 min. The bands corresponding to one and two ssDNA-conjugated gold nanoparticles were excised and eluted in 0.5×TBE buffer containing 0.5 mM bis(p-sulfonatophenyl)phenylphosphine dihydrate (BSPP). The purified one and two ssDNA-conjugated gold nanoparticles were hybridized at a concentration ratio of 2:1 at 37° C. overnight. The hybridized gold seeds were then separated by gel electrophoresis at 75 V for 25 minutes. The band containing the hybridized gold seeds was excised and eluted in a buffer solution. The purified gold seeds were concentrated by centrifugation at 14,000 rpm at 4° C. for 30 minutes. Then, the concentration was analyzed by UV-VIS spectroscopy. Thereafter, the gold seeds were mixed with mPEG-SH (Mw 2000) at a ratio of 1:100M and incubated overnight at 25° C. The gold seeds (0.5 nM) were reacted with a gold precursor (HAuCl₄, 0.03%) and a reducing agent (NH₂OH·HCl, 1 mM) at pH 5.0 and a NaCl concentration ranging from 50 mM to 100 mM for 1 hour and 3 hours, respectively. The morphology of the synthesized gold nanostructures was then observed by energy-filtering TEM. In addition, the nanostructure size and angle were analyzed using Image J software.

### Example 3: Preparation of Single Programmable Curved Plasmonic Nanoarchitecture Conjugated with Capture Probe

To conjugate the LNA capture probe to the nanoarchitecture surface, the 5′-end of the LNA capture probe was modified with a thiol group. The LNA capture probe was treated with a DTT solution for 15 minutes to break the disulfide bond, and the process of adding an equal amount of acetic acid and removing the supernatant was repeated three times. Then, the thiol group-activated LNA capture probe was incubated with the gold nanostructures of Example 2 at a ratio of 100:1 M at 25° C. overnight. The gold nanostructures conjugated with the LNA capture probe were centrifuged at 8000 rpm at 4° C. for 30 min. The pellet was then resuspended in DEPC-treated water.

### Example 4: Fabrication of Single Programmable Curved Plasmonic Nanoarchitecture (Nanostructure)-Based Biosensor

To fabricate a single nanoarchitecture-based sensor platform, the capture probe-conjugated gold nanoarchitecture solution prepared in Example 3 was diluted to an optical density of 0.02, and an MPTES-treated slide glass was drop-coated with 10 μL of the gold nanoarchitecture. The coated slide glass was fixed in a microfluidic imaging chamber. Thereafter, the impurities and nanostructures not bound to the slide surface were washed out with DEPC-treated water at a flow rate of 150 μL/min for 1 hour. Next, 200 μL of ethanolamine solution (0.1 M) was injected and incubated for 30 min to block the non-reactive surface of the capture probe-con jugated nanostructures. Excess ethanolamine molecules were washed out with DEPC-treated water at a flow rate of 150 μL/min for 5 min. Sequentially, 200 μL of human serum containing each target biomarker (concentration range of 10^{-0.5} aM-10^{9.5} aM) was injected at a flow rate of 20 μL/min. A mixed sample of exomiR and the LNA detection probe was incubated at 60° C. for 4 hours. Finally, the target biomolecule not bound to the capture probe on the nanostructure surface was washed out with DEPC-water at a flow rate of 150 μL/min for 5 min.

Thereafter, the biological changes of each single nanostructure surface were analyzed by Rayleigh scattering measurements at all stages. The spectra of the nanostructures were recorded under white light using a microscope and a CCD. The LSPR spectral changes were calculated using the Lorentz formula with the OriginPro 8 program.

### Example 5: Design of Programmable Curved Plasmonic Nanoarchitecture (Nanostructure) with Enhanced Optical Properties

The essential factor in the development of biosensors for clinical use is their ability to detect biomolecules. Therefore, it is necessary to construct a recognition transducer with improved performance capable of precisely and sensitively detecting target molecules. Because the unique physical and chemical properties of AuNPs can be easily tuned by changing geometrical and structural parameters, they are most often used as transducer components enabling efficient recognition and conversion processes.

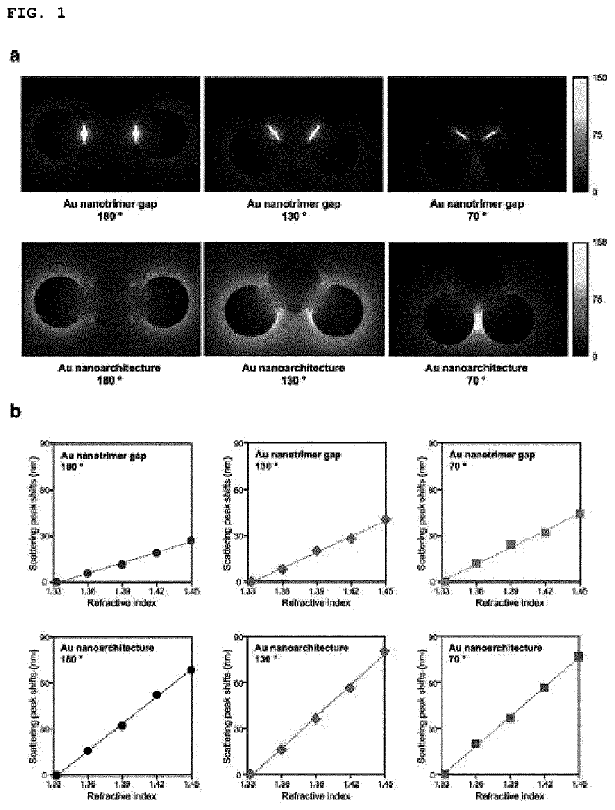

The optical properties of plasmonic metal structures can be greatly improved by introducing nanospaces such as nanogaps, nanocavities, and nanocrevices. These structures strongly amplify electromagnetic fields and concentrate incident light into subwavelength volumes that cause plasmon mode hybridization. Therefore, in the present invention, the nanostructure with improved spectral sensitivity was designed by introducing nanocrevices between nanospheres and additionally bending the nanostructure at 130° to create a single nanocavity. To further investigate the sensitivity of the plasmonic nanostructure, the electromagnetic field distributions and RI sensitivities of a nanotrimer composed of three close nanoparticles and the pre-designed structure were compared using FDTD simulation (FIG. 1).

As a result, as can be seen in FIG. 1, it could be confirmed that, compared to the trimer with a nanogap of 1 nm, the programmable curved plasmonic nanostructure with two narrow gaps formed a uniform and strong electromagnetic field distribution over the entire structure surface, and the RI sensitivity thereof increased by 2.50 times. This is believed to be because the nanogap (nanocrevis) generated a stronger electromagnetic field in the neck space due to charge transfer, thereby increasing the electron density and induced surface dipole, whereas the intergap generated a stronger electromagnetic field only ins the gap of the nanostructure. Then, the influence of the bending angle on the electromagnetic field and RI sensitivity in the nanocavity was examined. As a result, it was confirmed that the programmable curved plasmonic nanostructure with a 130° curved surface improved the electromagnetic field and RI sensitivity compared to the 180° and 70° curved nanostructures. Since the nanocavity inside the curved nanostructure confines the electromagnetic field into a subwavelength space, the electromagnetic field is generated more strongly in the curved nanostructure than in the linear structure (180°) that has only the intergap. In addition, the curved nanostructure causes the electric field of the incident light to circulate at the nanoparticle boundary, inducing a strong magnetic resonance in the adjacent space. This phenomenon is greatly affected by the nanogap size, and the space within the 70° curved nanostructure has the same size as the nanogap of the nanotrimer composed of three close nanoparticles, so the electromagnetic field amplification occurs relatively weakly compared to that in the 130° curved nanostructure. Based on these simulation results, it could be confirmed that the pre-designed programmable curved plasmonic nanoarchitecture (130°) with significantly improved electromagnetic field and RI sensitivity has sufficient potential for use as a biosensing material.

### Example 6: Synthesis and Characterization of Programmable Curved Plasmonic Nanostructure

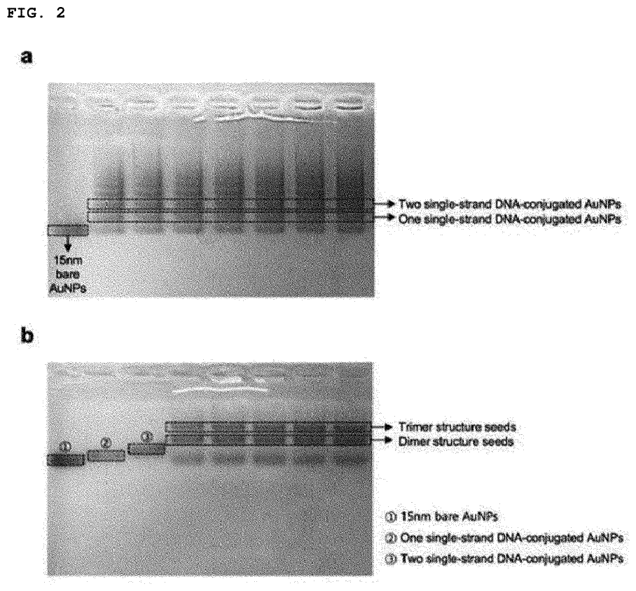

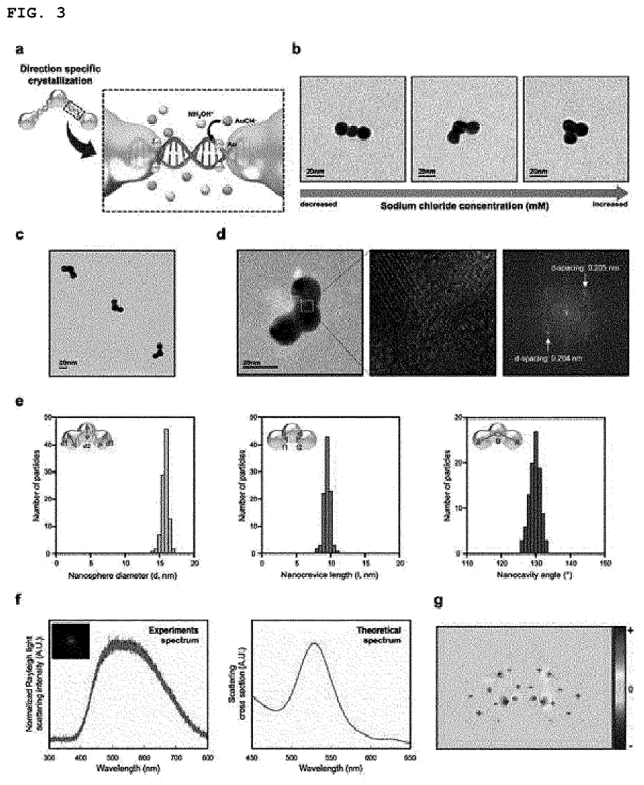

In order to synthesize the novel nanostructure pre-designed in Example 5, DNA-encoded gold (Au) seeds were prepared by gel electrophoresis (FIG. 2), and a synthesis process was performed based on a direction-specific crystallization technique, thereby performing precisely controlled crystallization of Au atoms based on double-stranded DNA (dsDNA) (FIG. 3a). In the metallization process of Au seeds, PEG and solution pH are important factors for the directional growth of gold ions along dsDNA. PEG coating increases the activation energy of the particle surface, allowing the crystallization reaction of the DNA surface to proceed faster than that of the nanoparticle surface. Since DNA has a slightly negative charge close to neutrality (isoelectric point (pI) of dsDNA=4.0 to 4.5) at pH 5, it maximizes the reducing agent effect and concentrates gold ions around DNA. Therefore, the crystallization of Au atoms starts at the nanoparticle-dsDNA interface and proceeds toward the center of dsDNA. The growth patterns of curved nanostructures with gaps including nanocrevis and nanocavities depending on the concentration of sodium chloride were analyzed by TEM, zeta potential, and dynamic light scattering (DLS). As a result, as can be seen in FIG. 3b, it could be confirmed that the distance between the nanoparticles at both ends of the trimer nanostructure became shorter as the concentration of sodium chloride increased (FIG. 3b). In addition, it could be confirmed that the surface charge of the DNA-encoded Au seeds changed from negative to neutral, and the size of the particles increased as the concentration of sodium chloride increased. This is because sodium chloride reduced the energy barrier generated by the surface charge of the gold nanoparticles.

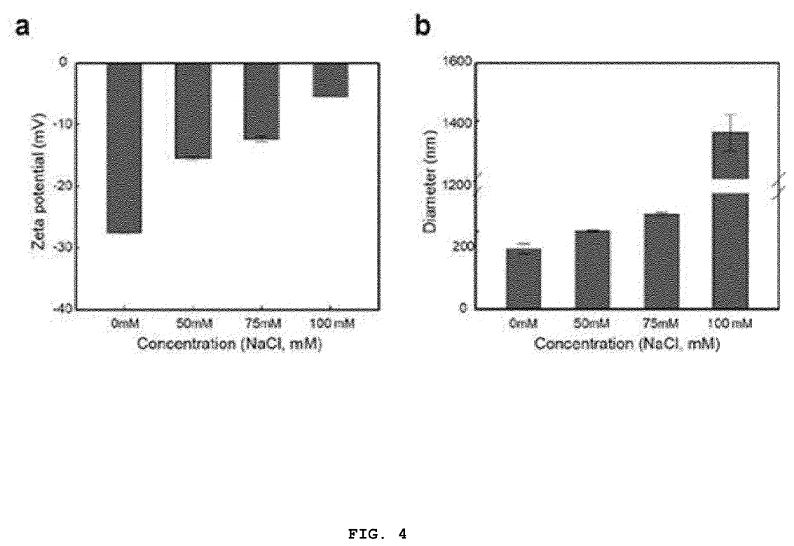

In addition, as can be seen in FIG. 4, sodium chloride enables repulsion between nanoparticles and the interaction between individual particles (FIG. 4). In particular, it could be confirmed that the pre-designed programmable curved plasmonic nanostructure with an internal bending angle of 130° was synthesized at a sodium chloride concentration of 75 mM (FIG. 3c).

Furthermore, the fast Fourier transform (FFT) diffraction pattern analysis showed that the lattice fringes corresponding to the crystal direction of the face-centered cubic (FCC) crystal structure of gold were identified, suggesting that gold crystallization successfully occurred in the DNA linkage region (FIG. 3d). In addition, the particle diameter was 15.82±0.50 nm, the nanogap was 9.49±0.49 nm, and the cavity bending angle was 129.70±1.57°, which were very similar to those of the pre-designed particles, indicating that the particles exhibited shape uniformity and size homogeneity (FIG. 3e). The yield of the curved plasmonic nanostructure was found to be 77.78%. In summary, it can be predicted that the nanostructure of the present invention will have high signal reproducibility as a sensing material.

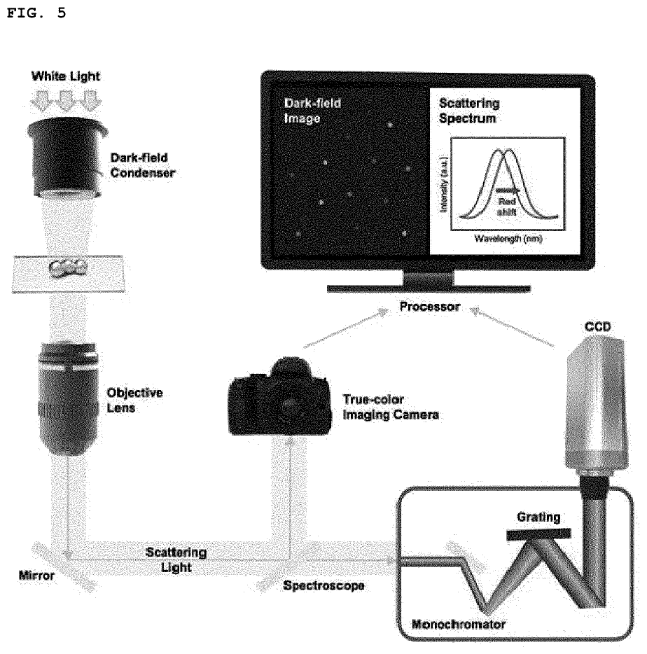

Meanwhile, in order to confirm that the nanostructure of the present invention is applicable as a basic material for an optical biosensor, resonant Rayleigh scattering in individual nanoarchitectures was measured by spectroscopy using a white light source (FIG. 5). As a result, it was confirmed that the programmable curved plasmonic nanostructure was displayed in yellow-orange color under a dark-field microscope. In addition, it was confirmed that the spectrum of the plasmonic nanostructure had a single uniform peak at 531.04±0.49 nm, which is consistent with the theoretical scattering spectrum result (λmax=529.0 nm) (FIG. 3f). In the present invention, only a single peak assigned to the bonding (σ) plasmon mode appeared in the scattering spectrum, and the peak of the antibonding (σ*) plasmon mode was not found due to the asymmetric nature of the programmable curved plasmonic nanostructure corresponding to the C2v point group. Unlike plasmonic hybridization between two metal nanoparticles forming σ and σ* plasmon modes, this asymmetric property also affects the unconventional charge distribution of the nanostructure, so that there is no clear distinction between positive and negative charges in any area (FIG. 3g). These results suggest that the pre-designed nanostructure can be fabricated with considerable precision using DNA-directed Au crystallization technology, and that the optical properties of the programmable curved plasmonic nanostructure can be tuned for intended purposes.

### Example 7: Fabrication and Performance Validation of Programmable Curved Plasmonic Nanoarchitecture-Based Plasmonic Biosensor for Alzheimer's Disease Diagnosis

Based on the programmable curved homogeneous nanostructure described above, the present inventors fabricated a clinical plasmonic biosensor capable of detecting four serum-derived exomiRs, including exomiR-125b, exomiR-135a, exomiR-15a, and exomiR-20a, as diagnostic markers for accurate Alzheimer's disease diagnosis. For reference, the exomiRs are upregulated in the blood of AD patients and are associated with Aβ metabolisms, such as acceleration of Aβ accumulation, Aβ plaque-induced inflammatory response, and neuronal death by regulation of amyloid precursor protein (APP)-related genes (e.g., APP, beta-secretase 1 (BACE1) and aph-1 homolog A (APH1A)).

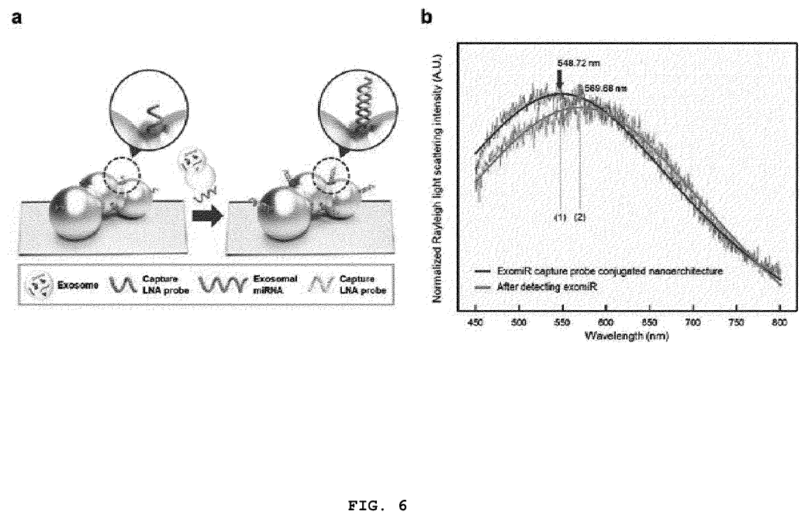

For target exomiR analysis with high sensitivity, selectivity, and binding affinity, the platform of the present invention enhanced Watson-Crick base pairing strength by sandwich hybridization between exomiR and the LNA (locked nucleic acid) capture/detection probe (FIG. 6a). To evaluate the validity of the sensor for miRNA detection, intact exomiR-125b was injected into the platform and LSPR signal generation was analyzed (FIG. 6b). As a result, as can be seen in FIG. 6b, a significant signal shift of 22.52±1.41 nm could be found, indicating that perfect binding occurred between the target exomiR and the two LNA probes, forming a stable sandwich structure. These results suggest that the biosensor system of the present invention can stably analyze target miRNAs, indicating that the platform of the present invention can perform AD-derived exomiR profiling and provide the same to patients.

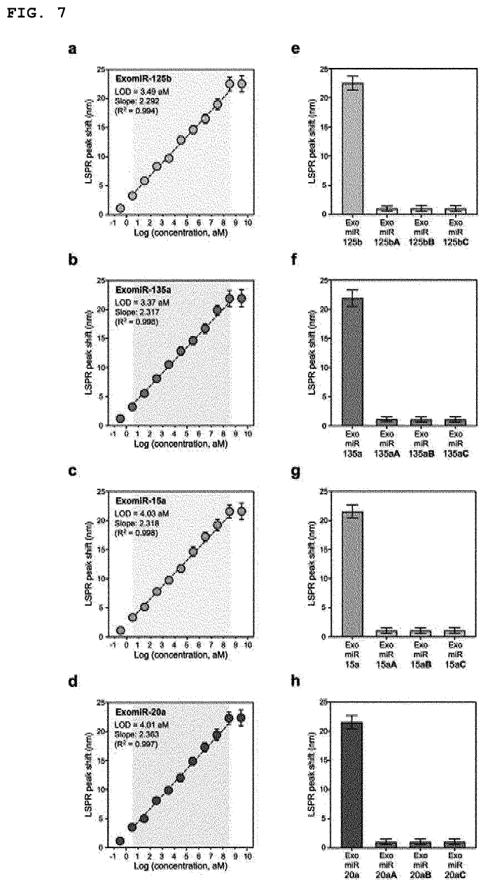

Meanwhile, in order to apply the biosensor of the present invention to clinical diagnosis, its ability to detect disease-related biomolecules with high sensitivity and selectivity is essential. In the biosensor system of the present invention, the LOD can be derived from the ratio between the LSPR signal and the target biomolecule concentration. In general, sensitivity is the ratio between the detected signal and the measured analyte and can be calculated as the slope of the system transfer equation. Here, the slope is used to obtain the sensor LOD, which is the lowest target analyte concentration that can be reliably detected. Therefore, the LOD of the sensor was determined by measuring the LSPR signal for each of the four AD biomarkers in mimicked serum over a range of concentrations. As a result, as can be seen in FIGS. 7a to 7d, it could be confirmed that, in the present invention, each of the four exomiR biomarkers (exomiR-125b, exomiR-135a, exomiR-15a, and exomiR-20a) showed a strong linear relationship with the LSPR shift at logarithmic concentrations ranging from 10^{0.5} to 10^{8.5} aM (FIGS. 7a to 7d). In addition, the coefficient of the slope (R2) was found to be 0.99 or higher. Furthermore, the LOD was calculated using the linear regression equation described in the online method, which supports the LOD from 3.37 to 4.01 for the four AD biomarkers. This value is 104 to 106 times lower than the number of AD biomarkers in patient blood samples. The extremely low LOD value in the biosensor of the present invention is considered to be possible due to the strong amplification of the electromagnetic field across the surface of the programmable curved plasmonic nanoarchitecture, as the nanogaps and the nanocavity effectively confine the incident light inside the structure.

In addition, as can be seen in Table 1 below, it was confirmed that the detection sensor of the biosensor of the present invention has improved sensitivity by at least 422 times compared to previously reported AD biomarker detection sensors (Table 1). These results demonstrate that the biosensor system of the present invention has the ability to sensitively and accurately detect exomiR biomarkers in clinical samples. Since human biological fluids, such as blood, serum, plasma, and urine, are rich in non-specific biomolecules, sensors applied in clinical settings must be rigorously evaluated for selectivity.

TABLE 1

| Method | Target | LOD | | Dynamic range | Sample type | Disease | References |
|---|---|---|---|---|---|---|---|
| LSPR | miR-125b | 3.49 | [symbol] M | 10 [symbol] M-10 [symbol] M | Serum | AD | |
| | miR-135a | 3.37 | [symbol] M | 10 [symbol] M-10 [symbol] M | | | |
| | miR-15a | 4.03 | [symbol] M | 10 [symbol] M-10 [symbol] M | | | |
| | miR-20a | 4.01 | [symbol] M | 10 [symbol] M-10 [symbol] M | | | |
| EIS | miR-137 | 1.70 | fM | 5 fM-750 fM | Serum | AD | (Dong et al. 2015) |
| FRET | miR-29a | 74.5 | pM | 0 nM-20 nM | Serum | AD | (Kim et al. 2020) |
| Fluorescence | miR-137 | 82 | pM | 0.05 nM-5 nM | Serum | AD | ( [symbol] and [symbol] 2019) |

| | |
|---|---|
| and GO | miR-142 |

*LSPR: Localized Surface Plasmon Resonance, EIS: Electrochemical Impedance Spectroscopy, FRET: Fluorescence Resonance Energy Transfer, GO: Graphene Oxide. [symbol]
indicates data missing or illegible when filed

In particular, due to the high sequence similarity among members of the miRNA family, one of the most important challenges in miRNA analysis for clinical diagnosis is to accurately distinguish between miRNAs of the same family with a single nucleotide difference. To this end, in the present invention, the selectivity of the programmable curved plasmonic nanoarchitecture-based plasmonic biosensor for target exomiRs was evaluated using three types of mimic samples (FIGS. 7e and 7h). As can be seen in Table 2 below, each of types A, B, and C has a missense mutation at the 3′ end, middle, and 5′ end of the target miRNA sequence (Table 2).

TABLE 2

| | | | Length | Tm |
|---|---|---|---|---|
| Assigned name | | Sequence (5′→3′) | (bp) | (° C.) |
| miRNA | miR-125b | UCCCUGAGACCCUAACUUGUGA | 22 | |
| | miR-125bA | UCCCUGAGACCCUAACUUGAGA | 22 | |
| | miR-125bB | UCGCUGAGACCCUAACUUGUGA | 22 | |
| | miR-125bC | UCCCUGAGACCGUAACUUGUGA | 22 | |
| | miR-135a | UAUAGGGAUUGGAGCCGUGGCG | 22 | |
| | miR-135aA | UAUAGGGAUUGGAGCCGUGCCG | 22 | |
| | miR-135aB | UAUAGGGAUUGCAGCCGUGGCG | 22 | |
| | miR-135aC | UAAAGGGAUUGGAGCCGUGGCG | 22 | |
| | miR-15a | UAGCAGCACAUAAUGGUUUGUG | 22 | |
| | miR-15aA | UAGCAGCACAUAAUGGUUUCUG | 22 | |
| | miR-15aB | UAGCAGCACAUUAUGGUUUGUG | 22 | |
| | miR-15aC | UACCAGCACAUAAUGGUUUGUG | 22 | |
| | miR-20a | UAAAGUGCUUAUAGUGCAGGUAG | 23 | |
| | miR-20aA | UAAAGUGCUUAUAGUGCAGGAAG | 23 | |
| | miR-20aB | UAAAGUGCUUAAAGUGCAGGUAG | 23 | |
| | miR-20aC | UAUAGUGCUUAUAGUGCAGGUAG | 23 | |
| LNA | miR-125b | Thiol-T[C]A[C]A[A][G]TT[A]G | 11 | 71 |
| capture | | | | |
| probe | | | | |
| | miR-135a | Thiol-C[G]CCACGGCTC | 11 | 73 |
| | miR-15a | Thiol-C[A][C]AAA[C][C]ATT | 11 | 73 |
| | miR-20a | Thiol-CTA[C]CT[G]CACT | 11 | 71 |
| LNA | miR-125b | GGT[C]TCAG[G]GA | 11 | 71 |
| detection | | | | |
| probe | | | | |
| | miR-135a | C[C]AAT[C]CTA[T]A | 11 | 72 |
| | miR-15a | AT[G]TG[C]T[G]CTA | 11 | 72 |
| | miR-20a | A[T]AA[G]C[A][C]TTTA | 12 | 71 |

Here, the LNA probe enhances the detection performance of the biosensor by forming a structure that specifically and selectively hybridizes with the target probe. As a result, as can be confirmed in FIGS. 7e to 7h, it was confirmed that only the samples containing each target exomiR showed an LSPR signal change greater than 20.00 nm. In contrast, it was confirmed that no clear LSPR shift occurred for miRNAs containing a single point mutation (less than 2.00 nm). These results demonstrate that the biosensor of the present invention detects specific miRNAs to distinguish single-nucleotide differences. Therefore, the biosensor of the present invention may be applied to changes in the severity and brain status of AD through exomiR signature expression profiling in real blood samples.

### Example 8: Detection of AD Biomarkers in Clinical Serum Samples

To verify the clinical applicability of the biosensor of the present invention, target biomarker detection was performed on clinical serum samples from three hospital-based cohorts including AD patients, MCI patients, and HCs (healthy controls) (detailed clinical information of the participants is shown in Table 3 below).

TABLE 3

| Patients | | | Eth- | | MMSE | | Diag- |
|---|---|---|---|---|---|---|---|
| number | Type | Gender | nicity | Age | score | CDR | nosis |
| KUMC-0050 | Serum | M | Asian | 33 | 30 | | HC |
| KUMC-0051 | Serum | M | Asian | 32 | 30 | | HC |
| KUMC-0054 | Serum | M | Asian | 31 | 30 | | HC |
| KUMC-0055 | Serum | M | Asian | 29 | 30 | | HC |
| KUMC-0056 | Serum | M | Asian | 30 | 30 | | HC |
| KUMC-0001 | Serum | M | Asian | 79 | 19 | 0.5 | AD |
| KUMC-0003 | Serum | F | Asian | 78 | 23 | 0.5 | AD |
| KUMC-0010 | Serum | F | Asian | 71 | 22 | 0.5 | AD |
| KUMC-0011 | Serum | M | Asian | 57 | 15 | 1 | AD |
| KUMC-0017 | Serum | M | Asian | 77 | 15 | 1 | AD |
| KUMC-0020 | Serum | F | Asian | 62 | 24 | 0.5 | AD |
| KUMC-0021 | Serum | F | Asian | 58 | 18 | 1 | AD |
| KUMC-0024 | Serum | F | Asian | 89 | 13 | NA | AD |
| KUMC-0025 | Serum | F | Asian | 67 | 2 | 3 | AD |
| KUMC-0026 | Serum | M | Asian | 64 | 17 | 1 | AD |
| KUMC-0029 | Serum | F | Asian | 70 | 26 | 0.5 | AD |
| KUMC-0030 | Serum | F | Asian | 75 | 24 | 0.5 | AD |
| KUMC-0031 | Serum | F | Asian | 72 | 25 | 0.5 | AD |
| KUMC-0035 | Serum | F | Asian | 67 | 23 | 0.5 | AD |
| KUMC-0037 | Serum | M | Asian | 78 | 22 | 0.5 | AD |
| KUMC-0040 | Serum | F | Asian | 77 | 20 | 0.5 | AD |
| KUMC-0041 | Serum | M | Asian | 65 | 23 | 0.5 | AD |
| KUMC-0042 | Serum | M | Asian | 67 | NA | NA | AD |
| KUMC-0045 | Serum | E | Asian | 77 | 26 | 0.5 | AD |
| KUMC-0049 | Serum | F | Asian | 66 | 18 | 0.5 | AD |
| KUMC-0062 | Serum | M | Asian | 84 | 27 | 0.5 | AD |
| KUMC-0067 | Serum | F | Asian | 66 | 22 | 0.5 | AD |
| KUMC-0069 | Serum | M | Asian | 78 | 26 | 0.5 | AD |
| KUMC-0070 | Serum | M | Asian | 83 | 26 | 0.5 | AD |
| KUMC-0004 | Serum | M | Asian | 63 | 28 | 0.5 | MCI |
| KUMC-0005 | Serum | M | Asian | 73 | 26 | 0.5 | MCI |
| KUMC-0006 | Serum | M | Asian | 62 | 27 | 0.5 | MCI |
| KUMC-0009 | Serum | F | Asian | 65 | 30 | 0.5 | MCI |
| KUMC-0013 | Serum | M | Asian | 63 | 25 | 0.5 | MCI |
| KUMC-0015 | Serum | F | Asian | 67 | 28 | 0.5 | MCI |
| KUMC-0018 | Serum | M | Asian | 79 | 21 | 0.5 | MCI |
| KUMC-0022 | Serum | M | Asian | 79 | 20 | 0.5 | MCI |
| KUMC-0023 | Serum | M | Asian | 79 | 26 | 0.5 | MCI |
| KUMC-0034 | Serum | M | Asian | 65 | 29 | 0.5 | MCI |
| KUMC-0039 | Serum | M | Asian | 66 | 29 | 0.5 | MCI |
| KUMC-0052 | Serum | F | Asian | 78 | 24 | 1 | MCI |
| KUMC-0057 | Serum | M | Asian | 70 | 29 | 0.5 | MCI |
| KUMC-0064 | Serum | M | Asian | 77 | 21 | 0.5 | MCI |

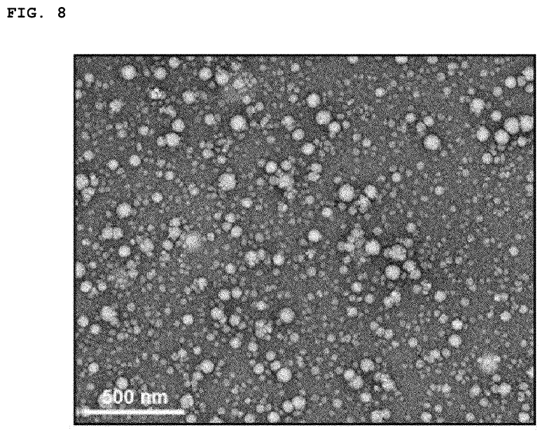

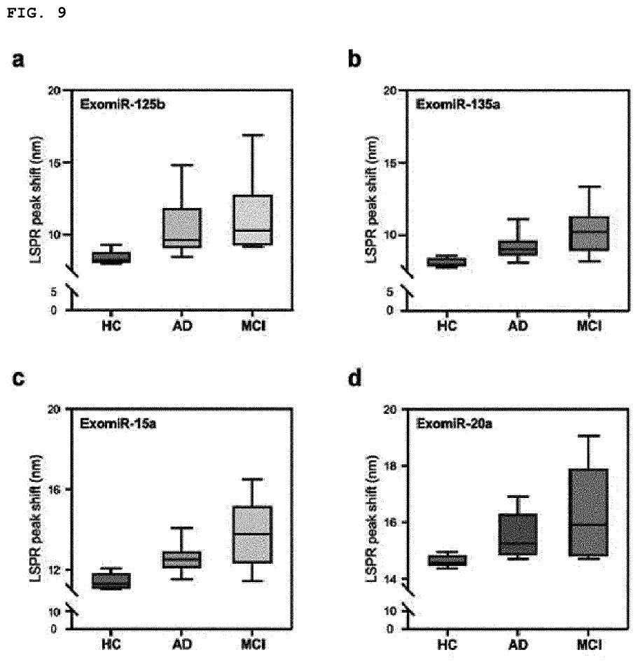

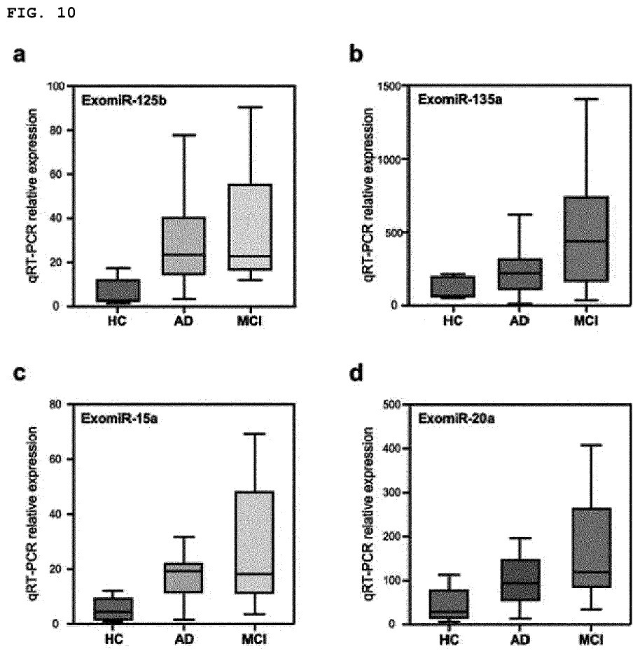

Before testing clinical samples, exosomes were isolated from serum for exomiR detection and their size and morphology were analyzed by TEM (FIG. 8). Then, exomiRs were extracted and analyzed for the average expression levels of AD biomarkers among three hospital-based cohorts using LSPR-based sensing. As a result, as can be shown in FIG. 9, it could be confirmed that the four exomiRs showed higher expression levels in AD and MCI patients than in HCs (FIG. 9). ExomiR-125b expression was 2.22-fold (t-test, p=0.0003) and 2.67-fold (t-test, p=0.0008) higher in AD and MCI patients, respectively, than in HCs. These results indicate that the expression patterns of these four AD biomarkers are well consistent with the results of the conventional analysis method of qRT-PCR (FIG. 10).

In order to evaluate the efficiency of dementia diagnosis based on a single Alzheimer's disease biomarker using the biosensor of the present invention, the AUC value was determined by receiver operating characteristic curve (ROC) analysis, which is a statistical method for evaluating the sensitivity, selectivity, and accuracy of a diagnostic tool (Table 4). Here, the AUC value is a method for quantifying the efficiency of the analysis method with a value closer to 1.0 indicating more accurate clinical prediction (Rifai et al., 2006).

TABLE 4

| | | Cut-off | Sensi- | Selec- | | |
|---|---|---|---|---|---|---|
| | | value | tivity | tivity | Accuracy | |
| | Biomarkers | (nm) | (%) | (%) | (%) | AUC |
| HC | Aβ 1-40 | 15.55 | 83.33 | 60.00 | 88.23 | 0.858 |
| vs | Aβ 1-42 | 12.32 | 62.50 | 60.00 | 71.43 | 0.629 |
| AD | ExomiR-125b | 8.62 | 91.67 | 80.00 | 97.78 | 0.908 |
| | ExomiR-135a | 8.39 | 87.50 | 80.00 | 96.55 | 0.933 |
| | ExomiR-15a | 11.80 | 91.30 | 80.00 | 97.67 | 0.948 |
| | ExomiR-20a | 14.75 | 91.67 | 80.00 | 97.78 | 0.900 |
| HC | Aβ 1-40 | 15.53 | 71.43 | 60.00 | 78.95 | 0.793 |
| vs | Aβ 1-42 | 12.31 | 50.00 | 60.00 | 60.00 | 0.571 |
| MCI | ExomiR-125b | 9.16 | 92.86 | 80.00 | 98.11 | 0.929 |
| | ExomiR-135a | 8.45 | 85.71 | 80.00 | 96.00 | 0.943 |
| | ExomiR-15a | 11.76 | 85.71 | 80.00 | 96.00 | 0.929 |
| | ExomiR-20a | 14.75 | 91.67 | 80.00 | 97.78 | 0.914 |
| AD | Aβ 1-40 | 15.64 | 57.14 | 54.17 | 61.18 | 0.525 |
| vs | Aβ 1-42 | 12.31 | 50.00 | 50.00 | 50.00 | 0.557 |
| MCI | ExomiR-125b | 9.89 | 57.14 | 58.33 | 65.11 | 0.594 |
| | ExomiR-135a | 9.13 | 64.29 | 62.50 | 75.00 | 0.699 |
| | ExomiR-15a | 12.84 | 71.43 | 69.57 | 85.11 | 0.677 |
| | ExomiR-20a | 15.48 | 57.14 | 54.17 | 61.18 | 0.580 |

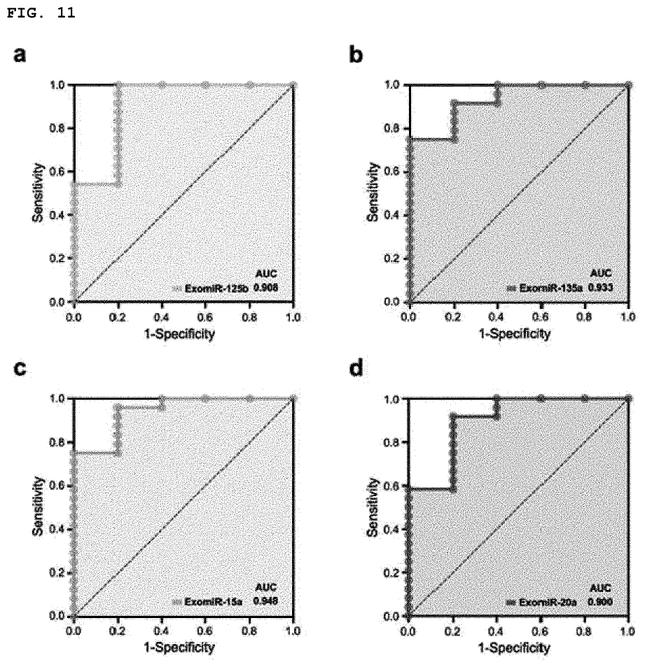

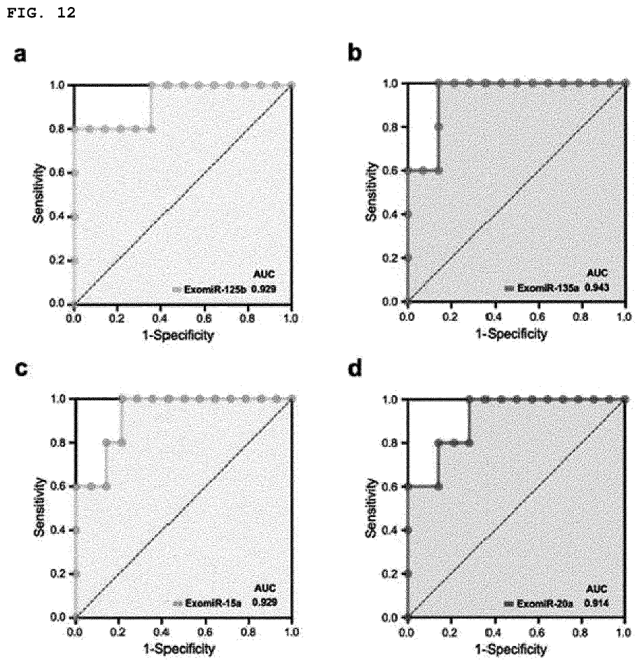

As a result, as can be seen in FIG. 6, it could be confirmed that the diagnosis of Alzheimer's disease using the four exomiR markers showed an AUC value of 0.90 or higher and an accuracy of 96.55% or higher, which were higher than those in the diagnosis using the Alzheimer's disease core biomarkers. These results indicate that not only the biosensor of the present invention has superior diagnostic performance compared to existing analysis methods, but also that the single exomiR biomarker-based diagnostic sensor system proposed in the present invention can accurately distinguish AD and MCI patients from HCs (FIGS. 11 and 12).

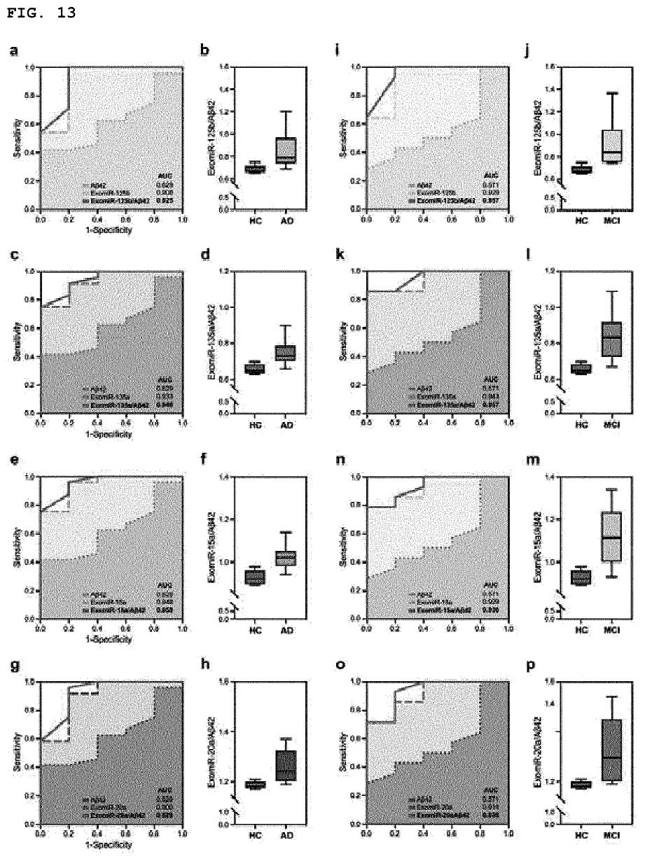

In addition, to increase the probability of distinguishing AD patients from HCs, the combination of core AD biomarker Aβ and exomiR was investigated. Specifically, the diagnostic performance was compared through ROC analysis using different combinations of AD biomarkers in serum between groups (Tables 5 and 6). As a result, as can be seen in FIG. 13, it could be confirmed that the detection of combinations of exomiRs and Aβ1-42 using the biosensor of the present invention could successfully distinguish between HC and AD patients with an average sensitivity of 95.83%, a selectivity of 80.0%, and an accuracy of 98.62%. In addition, it was confirmed that, when exomiR-125b and Aβ1-42 were applied as diagnostic markers, MCI patients could be diagnosed with a sensitivity of 100.0%, a selectivity of 80.0%, and an accuracy of 100.0%. The AUC values of the two clinical trial groups ranged from 0.936 to 0.957 (FIG. 13 and Table 5).

TABLE 5

| | | | Sensi- | Selec- | | |
|---|---|---|---|---|---|---|
| | | Cut-off | tivity | tivity | Accuracy | |
| | Biomarkers | value | (%) | (%) | (%) | AUC |
| HC | ExomiR-125b/ | 0.685 | 100.00 | 80.00 | 100.00 | 0.925 |
| vs | Aβ 1-42 | | | | | |
| AD | ExomiR-135a/ | 0.675 | 91.67 | 80.00 | 97.78 | 0.946 |
| | Aβ 1-42 | | | | | |
| | ExomiR-15/a | 0.945 | 95.83 | 80.00 | 98.92 | 0.958 |
| | Aβ 1-42 | | | | | |
| | ExomiR-20a/ | 1.195 | 95.83 | 80.00 | 98.92 | 0.929 |
| | Aβ 1-42 | | | | | |
| HC | ExomiR-125b/ | 0.710 | 100.00 | 80.00 | 100.00 | 0.957 |
| vs | Aβ 1-42 | | | | | |
| MCI | ExomiR-135a/ | 0.685 | 85.71 | 80.00 | 96.00 | 0.957 |
| | Aβ 1-42 | | | | | |
| | ExomiR-15a/ | 0.955 | 92.86 | 80.00 | 96.00 | 0.936 |
| | Aβ 1-42 | | | | | |
| | ExomiR-20a/ | 1.195 | 92.86 | 80.00 | 98.11 | 0.936 |
| | Aβ 1-42 | | | | | |
| AD | ExomiR-125b/ | 0.795 | 64.29 | 58.33 | 71.59 | 0.604 |
| vs | Aβ 1-42 | | | | | |
| MCI | ExomiR-135a/ | 0.750 | 64.29 | 66.67 | 78.27 | 0.704 |
| | Aβ 1-42 | | | | | |
| | ExomiR-15a/ | 1.045 | 71.43 | 70.83 | 85.86 | 0.682 |
| | Aβ 1-42 | | | | | |
| | ExomiR-20a/ | 1.265 | 57.14 | 58.33 | 65.11 | 0.592 |
| | Aβ 1-42 | | | | | |

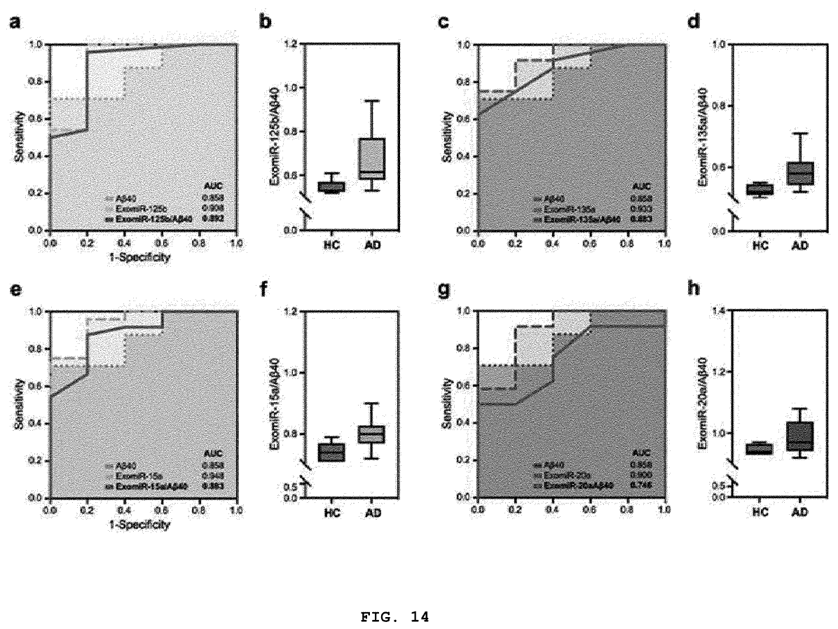

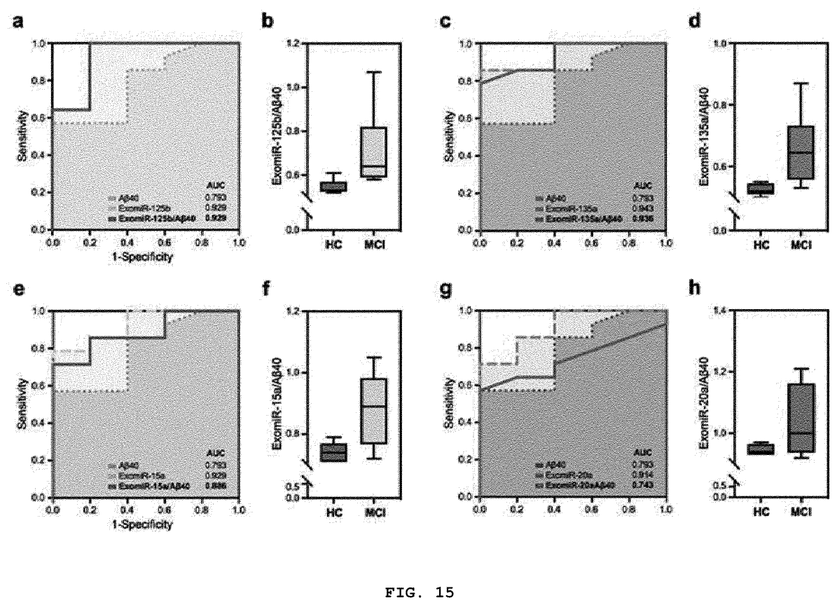

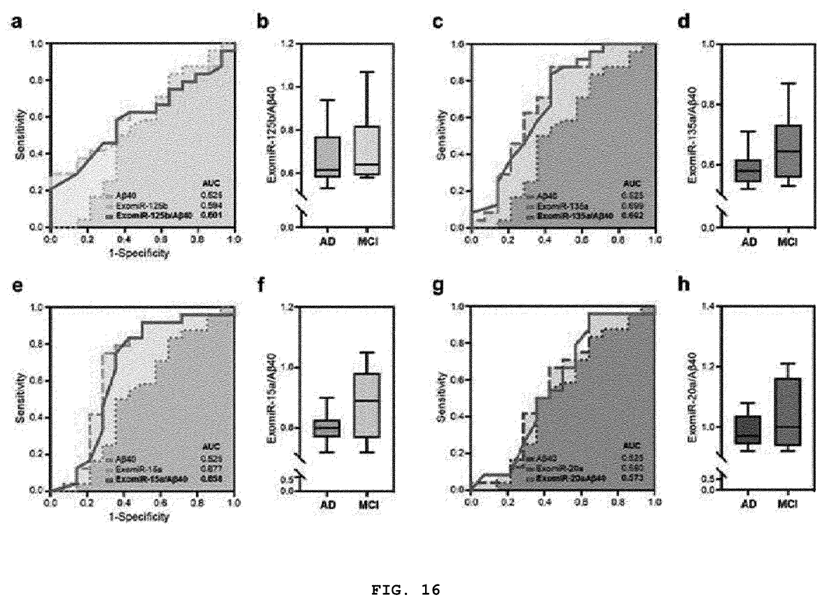

These results demonstrate that the biosensor of the present invention has improved diagnostic efficiency compared to the core Alzheimer's disease biomarker-based diagnostic approach, which is currently considered the most accurate in the diagnosis of Alzheimer's disease (Li et al., 2022; Verberk et al., 2020). On the other hand, the diagnostic accuracy of the exomiR/Aβ1-40 combination in dementia patients was evaluated to be lower than that of a single exomiR (Table 6 and FIGS. 14 to 16).

TABLE 6

| | | | Sensi- | Selec- | | |
|---|---|---|---|---|---|---|
| | | Cut-off | tivity | tivity | Accuracy | |
| | Biomarkers | value | (%) | (%) | (%) | AUC |
| HC | ExomiR-125b/ | 0.550 | 87.50 | 80.00 | 96.55 | 0.892 |
| vs | Aβ 1-40 | | | | | |
| AD | ExomiR-135a/ | 0.535 | 87.50 | 60.00 | 91.31 | 0.883 |
| | Aβ 1-40 | | | | | |
| | ExomiR-15a/ | 0.745 | 91.67 | 60.00 | 94.29 | 0.883 |
| | Aβ 1-40 | | | | | |
| | ExomiR-20a/ | 0.945 | 75.00 | 60.00 | 81.82 | 0.746 |
| | Aβ 1-40 | | | | | |
| HC | ExomiR-125b/ | 0.585 | 85.71 | 80.00 | 96.00 | 0.929 |
| vs | Aβ 1-40 | | | | | |
| MCI | ExomiR-135a/ | 0.545 | 85.71 | 80.00 | 96.00 | 0.936 |
| | Aβ 1-40 | | | | | |
| | ExomiR-15a/ | 0.755 | 85.71 | 80.00 | 96.00 | 0.886 |
| | Aβ 1-40 | | | | | |
| | ExomiR-20a/ | 0.945 | 71.43 | 60.00 | 78.95 | 0.743 |
| | Aβ 1-40 | | | | | |
| AD | ExomiR-125b/ | 0.625 | 64.29 | 58.33 | 71.59 | 0.601 |
| vs | Aβ 1-40 | | | | | |
| MCI | ExomiR-135a/ | 0.585 | 64.29 | 58.33 | 71.59 | 0.692 |
| | Aβ 1-40 | | | | | |
| | ExomiR-15a/ | 0.810 | 64.29 | 66.67 | 78.27 | 0.658 |
| | Aβ 1-40 | | | | | |
| | ExomiR-20a/ | 0.985 | 57.14 | 54.17 | 61.18 | 0.573 |
| | Aβ 1-40 | | | | | |

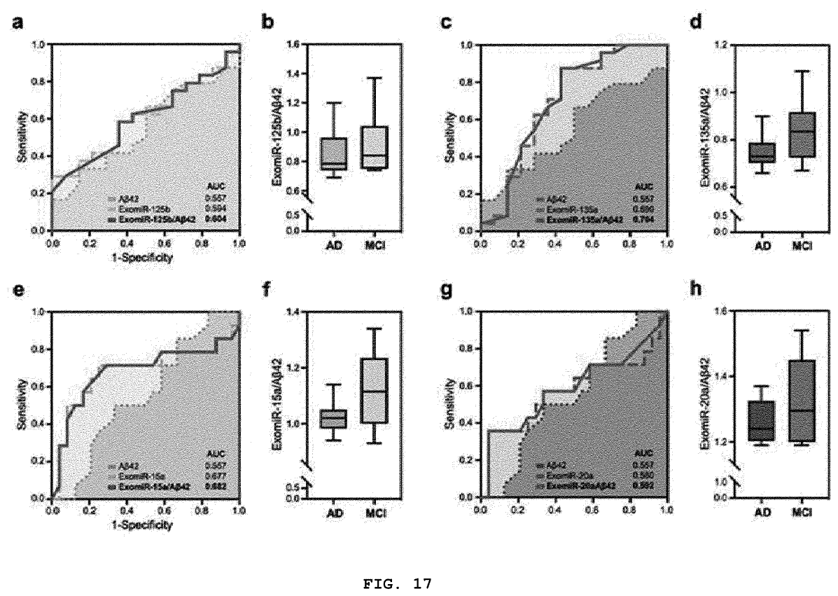

Since MCI has a high likelihood of developing into severe dementia, the ability of the biosensor of the present invention to accurately classify MCI and AD patients may be a means for achieving early diagnosis of dementia and providing effective treatment to patients. In this regard, the present inventors evaluated the diagnostic performance of the biosensor of the present invention for distinguishing between MCI and AD patients based on exomiR/AB combination biomarkers. In particular, the diagnostic classification accuracy was 85.86% (sensitivity=71.43%, selectivity=70.83%, and AUC=0.682) based on the combination of exomiR-15a and Aβ1-42 (FIG. 17). Therefore, the blood-based curved plasmonic biosensor of the present invention can be used not only as a promising alternative diagnostic technology for CSF-based analysis, but also as an analytical tool for recognizing dementia before serious pathophysiological changes occur.

In summary, the present invention proposes a programmable curved plasmonic nanoarchitecture-based biosensor for the clinical diagnosis of Alzheimer's disease. The pre-designed nanoarchitecture is implemented through simulation by introducing a nano-space (e.g., nanocrevice or nanocavity) that generates a strong electromagnetic field and then performing DNA-directed crystallization technology with a controlled bending angle. This strategy enables the synthesis of advanced optical nanostructures with various shapes and close-to-ideal physicochemical properties by tuning the structure at the nanometer level. In terms of RI sensitivity, the programmable curved nanostructure of the present invention is characterized by having a higher RI sensitivity level than other structures developed previously, and thus may be utilized as an excellent biosensor signal transducer. Thus, the newly synthesized nanoarchitecture-based plasmonic biosensor enables the detection of serum-derived ExomiRs that directly reflect AD-related pathological processes at the attomolar level. In addition, biomarker analysis based on hybridization between miRNA and the LNA probe distinguishes single-nucleotide differences between members of the miRNA family, allowing the sensor to accurately recognize specific target exomiRs. In addition, as a result of evaluating the validity of the curved nanoarchitecture-based plasmonic biosensor for clinical applications, the curved plasmonic biosensor of the present invention successfully classified clinically diagnosed AD, MCI patients, and HCs with an accuracy of 97.21% or higher by profiling exomiRs, including exomiR-125b, -135a, -15a, and -20a. Furthermore, the present inventors confirmed that the diagnostic performance for classifying AD and MCI patients from HC subjects can be improved by integrating the analyzed expression patterns of multiple exosome biomarkers. Therefore, it can be seen that the programmable curved plasmonic nanoarchitecture-based biosensor proposed in the present invention will be a promising tool for early diagnosis of dementia by elucidating the neuropathology of dementia through miRNA profiling in the future.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### INDUSTRIAL APPLICABILITY

The present invention is capable of detecting neurodegenerative disease markers present in blood with high selectivity and sensitivity, and thus may be effectively applied to the diagnosis of related diseases and in clinical fields.

1. A curved plasmonic metal nanostructure comprising a first metal nanoparticle, a second metal nanoparticle, and a third metal nanoparticle,
wherein the metal nanoparticles are arranged so that an angle between a first imaginary straight line connecting a midpoint of the second metal nanoparticle to a midpoint of the first metal nanoparticle and a second imaginary straight line connecting a midpoint of the second metal nanoparticle to a midpoint of the second metal nanoparticle is 125 to 135°, so that a nanocavity surrounded by the first metal nanoparticle, the second metal nanoparticle, and the third nanoparticle is formed, and
nanogaps are formed between the first metal nanoparticle and the second metal nanoparticle and between the second metal nanoparticle and the third metal nanoparticle, respectively.

2. The curved plasmonic metal nanostructure of claim 1, wherein the nanogaps have a diameter of 8 to 10 nm.

3. The curved plasmonic metal nanostructure of claim 1, wherein the metal nanoparticles have a diameter of 13 to 20 nm.

4. The curved plasmonic metal nanostructure of claim 1, wherein the second metal nanoparticle has single-stranded DNAs conjugated to both sides of the nanoparticle, and the first and third metal nanoparticles have conjugated thereto a single-stranded DNA complementary to the single-stranded DNA conjugated to the second metal nanoparticle.

5. (canceled)

6. The curved plasmonic metal nanostructure of claim 1, wherein the metal is any one selected from the group consisting of gold (Au), silver (Ag), copper (Cu), platinum (Pt), and palladium (Pd).

7. A curved plasmonic metal nanostructure-based biosensor comprising:
a substrate;
the curved plasmonic metal nanostructure of claim 1 immobilized on the substrate and having a capture probe conjugated thereto that specifically binds to a target biomarker; and
a measurement unit configured to measure a localized surface plasmon resonance phenomenon in the curved plasmonic metal nanostructure.

8. The curved plasmonic metal nanostructure-based biosensor of claim 7, wherein the target biomarker is any one or more selected from the group consisting of DNA, miRNA, a peptide, and a protein.

9. The curved plasmonic metal nanostructure-based biosensor of claim 8, wherein the miRNA is derived from exosomes.

10. The curved plasmonic metal nanostructure-based biosensor of claim 9, wherein the miRNA is exo-miR125b, exo-miR135a, exo-miR15a, exo-miR20a, or a combination thereof.

11. The curved plasmonic metal nanostructure-based biosensor of claim 9, wherein the biosensor detects exosome-derived miRNA and protein by measuring a Rayleigh scattering spectral change caused by specific binding of the exosome-derived miRNA and protein.

12. The curved plasmonic metal nanostructure-based biosensor of claim 11, wherein the biosensor has no change in the Rayleigh scattering spectrum when exo-miR125b, exo-miR135a, exo-miR15a or exo-miR20a has a single point mutation.

13. The curved plasmonic metal nanostructure-based biosensor of claim 8, wherein the protein is amyloid β (Aβ).

14. (canceled)

15. (canceled)

16. (canceled)

17. The curved plasmonic metal nanostructure-based biosensor of claim 7, wherein the capture probe comprises DNA, LNA, or an antibody.

18. A method for detecting an exosomal miRNA and protein derived from a neurodegenerative disease, comprising a step of treating the biosensor of claim 7 with a neurodegenerative disease-derived biomarker mixture.

19. The method of claim 18, wherein the biomarker mixture is blood, or serum from which agglutinins have been removed.

20. The method of claim 18, wherein the neurodegenerative disease is Alzheimer's disease or mild cognitive impairment.

21. The method of claim 18, wherein the exosomal miRNA is any one or more selected from the group consisting of exo-miR125b, exo-miR135a, exo-miR15a, and exo-miR20a.

22. The method of claim 18, wherein the protein is amyloid β.

23. A method for diagnosing a neurodegenerative disease, comprising steps of:
treating the biosensor of claim 7 with a neurodegenerative disease-derived biomarker mixture; and
treating the biosensor with an exosome-derived miRNA or protein detection probe.

24. The method of claim 23, wherein the detection probe comprises DNA or LNA.

25. (canceled)

26. (canceled)

27. (canceled)

28. (canceled)

29. (canceled)

30. (canceled)

31. (canceled)

32. (canceled)

33. (canceled)

34. (canceled)
